(19) United States
(12) Patent Application Publication
(10) Pub. No.: US 2011/0094415 A1
(43) Pub. Date: Apr. 28, 2011

(54) DENTURE ADHESIVE ARTICLES

(76) Inventors: Jayanth Rajaiah, Loveland, OH (US); Franco Silva Medeiros, Loveland, OH (US); Luisa Navarro Cerda, Cincinnati, OH (US); Robert Scott Leonard, Fairfield, OH (US); Steven Daryl Smith, Fairfield, OH (US); Mark William Hamersky, Indian Springs, OH (US); Rafael Edmundo Bras, West Chester, OH (US)

(21) Appl. No.: 12/939,422

(22) Filed: Nov. 4, 2010

Related U.S. Application Data
(63) Continuation-in-part of application No. 12/542,461, filed on Aug. 17, 2009.
Application No. 12/542,461 is a continuation-in-part of application No. 11/590,233, filed on Oct. 31, 2006, now abandoned.
Application No. 11/590,233 is a continuation-in-part of application No. 11/590,111, filed on Oct. 31, 2006, now abandoned.
Application No. 11/590,111 is a continuation-in-part of application No. 11/590,191, filed on Oct. 31, 2006, now abandoned.
Application No. 11/590,191 is a continuation-in-part of application No. 11/590,232, filed on Oct. 31, 2006, now abandoned.
Application No. 11/590,232 is a continuation-in-part of application No. 11/590,231, filed on Oct. 31, 2006, now abandoned.
Application No. 11/590,231 is a continuation-in-part of application No. 12/476,352, filed on Jun. 2, 2009.
Application No. 12/476,352 is a continuation-in-part of application No. 11/590,224, filed on Oct. 31, 2006, now abandoned.
(60) Provisional application No. 60/735,243, filed on Nov. 9, 2005.
(60) Provisional application No. 60/760,526, filed on Jan. 20, 2006.
(60) Provisional application No. 60/735,088, filed on Nov. 9, 2005.
(60) Provisional application No. 60/760,660, filed on Jan. 20, 2006.
(60) Provisional application No. 60/735,136, filed on Nov. 9, 2005.
(60) Provisional application No. 60/760,528, filed on Jan. 20, 2006.
(60) Provisional application No. 60/735,135, filed on Nov. 9, 2005.
(60) Provisional application No. 60/760,516, filed on Jan. 20, 2006.
(60) Provisional application No. 60/734,874, filed on Nov. 9, 2005.
(60) Provisional application No. 60/760,527, filed on Jan. 20, 2006.
(60) Provisional application No. 60/760,711, filed on Jan. 20, 2006.

(51) Int. Cl. A61K 6/083; A61K 6/08; A61Q 11/00

(52) U.S. Cl. 106/35; 525/207; 525/221; 525/55; 523/120; 525/222

(57) ABSTRACT

A denture adhesive article, comprising a denture adhesive component and a viscosity index improver, wherein the denture adhesive article is preformed.

### CROSS REFERENCE TO RELATED APPLICATION

This application is a continuation-in-part of U.S. application Ser. No. 12/542,461, filed Aug. 17, 2009, which is a continuation-in-part of U.S. application Ser. No. 11/590,233, filed Oct. 31, 2006, U.S. application Ser. No. 11/590,111, filed Oct. 31, 2006, U.S. application Ser. No. 11/590,191, filed Oct. 31, 2006, U.S. application Ser. No. 11/590,232, filed Oct. 31, 2006, and U.S. application Ser. No. 11/590,231, filed Oct. 31, 2006, which claimed the benefit of U.S. Provisional Application Nos. 60/735,243 filed Nov. 9, 2005; 60/760,526 filed Jan. 20, 2006; 60/735,088 filed Nov. 9, 2005; 60/760,660 filed Jan. 20, 2006; 60/735,136 filed Nov. 9, 2005; 60/760,528 filed Jan. 20, 2006; 60/735,135 filed Nov. 9, 2005; 60/760,516 filed Jan. 20, 2006; 60/734,874 filed Nov. 9, 2005 and 60/760,527 filed Jan. 20, 2006, and 60/760,711 filed Jan. 20, 2006 the substances of which are incorporated herein by reference.

This application is a continuation-in-part also of U.S. application Ser. No. 12/476,352, filed Jun. 2, 2009, which is a continuation-in-part of U.S. application Ser. No. 11/590,224, filed Oct. 31, 2006, the substance of which is incorporated herein by reference.

### TECHNICAL FIELD

This invention relates to denture adhesive articles and in particular to improved denture adhesive methods and articles which include an adhesive component and a viscosity index improver.

### BACKGROUND OF THE INVENTION

Ordinary removable dentures, dental plates and the like, comprise teeth mounted in a suitable plate or base. While dentures are traditionally fitted for the individual user, the fit can change over time which may result in slippage or discomfort. Whether the fit is good or bad, some users prefer extra security against slippage and/or dislodgement. Denture adhesives are used to temporarily adhere the dentures to the surfaces of the oral cavity, in particular the oral mucosa and give wearers the extra security they prefer. Denture adhesives are typically applied to the denture, oral surface, or both at the beginning of the day when the dentures are placed into the oral cavity. Unfortunately, denture adhesives tend to bioerode during the course of the day due to the action of saliva, chewing, drinking, and the like. This erosion leads to loss of adhesiveness, oozing of the adhesive into the oral cavity, dislodgement of the denture, food lodging under the denture, etc. As such, there is a need for improved denture adhesives.

### SUMMARY OF THE INVENTION

According to one embodiment, the present invention is directed to a denture adhesive article, comprising a denture adhesive component and a viscosity index improver, wherein the denture adhesive article is preformed.

In another embodiment, the present invention is directed to a denture adhesive article, comprising (a) from about 10.0% to about 60.0% of a denture adhesive component comprising AVE/MA, salts of AVE/MA, mixed salts of AVE/MA, or a combination thereof; and (b) from about 10% to about 90% of a viscosity index improver comprising microcrystalline wax, wherein the denture adhesive article is preformed.

In another embodiment, the present invention is directed to a method of manufacturing a denture adhesive composition, the composition comprising: a) from about 10.0% to about 60.0% of a denture adhesive component comprising AVE/MA, salts of AVE/MA, mixed salts of AVE/MA, carboxymethylcellulose, or a combination thereof; and b) from about 10.0% to about 90.0% of a viscosity index comprising microcrystalline wax, polyethylene, rubber, elastomers, and combinations thereof; the method steps comprising: i. mixing at least one of the denture adhesive components with the viscosity index improver at a temperature above about 10° C. below the melting point of the viscosity index improver; and ii. subsequently cooling the composition to about 10° C. below the melting point of the viscosity index improver.

These and other embodiments of the present invention will be more fully understood in light of the detailed description below.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings in which:

FIG. 1 is a top plain view of an embodiment of a concave shaped denture adhesive article having symmetrical dimensions;

FIG. 2 is a top plain view of an embodiment of a concave shaped denture adhesive article having asymmetrical dimensions.

FIG. 3 is a % water uptake chart.

FIGS. 4-6 are three views of a spindle shaft.

FIGS. 7-9 are three views of a spindle spacer.

FIGS. 10-12 are three views of a spindle endcap.

### DETAILED DESCRIPTION OF THE INVENTION

A detailed description of embodiments of the present invention is given below.

### DEFINITIONS

The abbreviation “cm”, as used herein, means centimeter. The abbreviation “mm” as used herein, means millimeter. The abbreviation “g” as used herein, means gram. The abbreviation “P” as used herein, means Pascal. The abbreviation “s” as used herein means second. The abbreviation “Ps” as used herein means Pascal—second. The abbreviation “oz” as used herein, mean ounce.

The term “denture” as used herein refers to either the upper or lower denture, or both.

The term “viscosity index improver” as used herein refers to a material which makes the viscosity and/or rheological properties of a material into which it is incorporated more stable as its temperature is increased over a defined range. In the case of denture adhesive products, the defined range is between about 25° C. and about 60° C.

The term “denture adhesive article” and/or “article” as used herein refers to articles designed to fit, conform and adhere to contoured surfaces, such as a denture, as well as the gums or the roof of the mouth. The articles herein are substantially solid prior to use and can be picked up manually in substantially one piece and positioned on the denture. They are also preformed, that is, they are pre-shaped and ready to be applied.

The term “dispensed/dispensable from a tube” as used herein refers to a composition which can be dispensed from a small denture adhesive tube under manual pressure. A small denture adhesive tube is made of a foil laminate, is about 3.5 inches long, about 0.48 inches wide, and holds about 0.25 oz of product. The internal diameter of the nozzle on the small denture adhesive tube is about 0.19 inches and the nozzle length is about 0.38 inches. An example of a small denture adhesive tube is a 0.25 oz sample size tube which is supplied by Alcan Corporation as stock item number 2293.

The term “flexible” or “flexible article” as used herein means that a 0.67 mm thick piece of the article may be wrapped 180 degrees around a solid cylinder of 1 cm diameter without cracking upon visual observation.

The term “safe and effective adhesive amounts” as used herein means an amount sufficient to provide adherence to the oral cavity and/or provide adherence of a denture to the oral cavity, without toxicity to the user or damage to oral tissue.

By “safe and effective amount”, as used herein, is meant an amount of an agent high enough to significantly (positively) modify the condition to be treated or positively modify the benefit sought, but low enough to avoid serious side effects (at a reasonable benefit/risk ratio), within the scope of sound medical/dental judgment. The safe and effective amount of an agent may vary with the particular condition being treated, the age and physical condition of the patient being treated, the severity of the condition, the duration of treatment, the nature of concurrent therapy, the specific form of the source employed, and the particular vehicle from which the agent is applied.

The term “AVE/MA” as used herein refers to alkyl vinyl ether-maleic acid or anhydride copolymer. The term “mixed polymer salts” or “mixed salts”, as used herein, refers to salts of AVE/MA where at least 2 different cations are mixed on the same polymer with each other or with other salts.

The term “free acid” or “FA” component, as used herein, refers either to the unreacted carboxyl groups (—COOH) of AVE/MA copolymer plus any other monovalent cations of carboxyl groups (e.g., COONa) of the polymer. Monovalent cations include Group IA cations, such as sodium, potassium, hydrogen, etc. In one embodiment, the term “free acid” refers to the unreacted carboxyl groups (—COOH) of AVE/MA plus sodium and potassium cations. In another embodiment, the term “free acid” refers only to the unreacted carboxyl groups (—COOH) of the AVE/MA.

The term “toxicologically-acceptable”, as used herein, is used to describe materials that are suitable in their toxicity profile for administration to humans and/or animals.

The term “nonaqueous”, as used herein, means that the article is substantially free of added water. Substantially free means that no free water is added to the article, but the article may contain about 5% or less of water that comes in as part of other components.

The term “water-insoluble”, as used herein, refers to a material that, when exposed to water, does not dissolve, but may disperse to varying degrees. Generally, a material is water-insoluble if it is less than about 10% soluble in water.

The term “thermoplastic” as used herein refers to a material that melts, softens, becomes more flexible, extrudable, deformable, shapable, moldable, flowable, processable, and/or changes rheology when exposed to heat. In one embodiment the material generally solidifies, hardens, and/or substantially returns to its original condition, when subsequently cooled.

The term “bioerodible”, as used herein, means that the article, when exposed to water or saliva, will erode over time due to physical and/or chemical action. The time necessary to erode the article can be any length of time from instantaneous to five days. The article may erode completely or substantially, however ultimately the article will lose its original form and/or integrity. For example, after application and use for at least about 24 hours in the oral cavity the article will not have sufficient product integrity to easily separate or peel, in its original form, from the denture or oral surface. In another embodiment some portion or residue from the article remains on the denture or oral surface after removing the denture from the oral cavity; however, this portion or residue from the article can be cleaned by brushing away with a toothbrush, but not easily separated from the denture.

The term “viscosity”, as used herein, refers to the internal resistance to flow or deformation of a material. This can be measured by the ratio of shearing stress to rate of shear; and in some embodiments where this ratio is not suitably measured, suitable rheological parameters such as loss modulus G″ or storage modulus G′ can be measured.

The percentages used herein to describe the cationic salt function of the alkyl vinyl ether-maleic acid or anhydride copolymers are defined as the stoichiometric percent of the total initial carboxyl groups reacted on the polymer.

Unless otherwise noted, the term “melting point” as used herein refers to the Drop Melting Point which is the temperature at which the material becomes sufficiently fluid to drop from the thermometer used in making the determination under prescribed conditions as listed in ASTM D-127. If ASTM D-127 is not suitable for the material in question, then ASTM D-3954 can be used instead.

Unless otherwise noted, the term “derivative” as used herein refers to when the primary polymeric backbone is left unchanged, but the side groups/chains and/or end groups are changed.

As used herein, the term “silicone” refers to siloxane polymers based on a structure of alternate silicon and oxygen atoms with various organic radicals attached to the silicon.

All other percentages used herein are by weight of the composition unless otherwise indicated.

All measurements referred to herein are made at 25° C. unless otherwise specified.

### Denture Adhesive Articles and Methods

Denture adhesives have become a daily product for many people who are looking for better fit and/or more security when wearing dentures. This has driven consumer demand for products which have improved properties like long-lasting hold, for example. The present denture adhesive articles deliver improvements on such desirable properties.

Denture adhesive cream formulations have heretofore been comprised mainly of natural or synthetic polymer materials suspended in an anhydrous oleagenous vehicle system comprising mineral oil and petrolatum. The petrolatum is added to thicken the formulation consistency to that of a cream which is extrudable from tubes. These formulations necessarily must be thick to prevent syneresis or phase separation because the solid adhesive particles are merely suspended in the oily vehicle. This thickness of the formulations makes them somewhat difficult to squeeze out from the tube. Additionally, although these formulations are fairly thick in consistency at ambient room temperature of about 25° C., they are not thermally very stable and hence tend to thin out even at slightly elevated temperatures. For example, at the body/mouth temperature of 37° C., at which these formulations are used, they tend to thin out and become runny and therefore ooze out from under the denture during use. The phenomenon is naturally further aggravated when hot liquids and foods are consumed by denture wearers who use such denture adhesive products. This problem with oozing of denture adhesive from under the dentures into the mouth is considered to be one of the major drawbacks to the consumer due to the unpleasant taste and mouth feel. Additionally, the holding property of the formulation is reduced due to the oozing or loss of product from under the denture. Oil separation is an additional drawback to such denture creams.

In general, the denture adhesive articles of the present invention include an adhesive component and a viscosity index improver. Historically, viscosity index improver was a term associated with the lubricant industry. The viscosity of a lubricant is closely related to its ability to reduce friction. The most desirable lubricant is one which will allow the easiest movement of two surfaces while still forcing the two moving surfaces apart, because this results in the lowest friction. However, as the viscosity of liquids tends to decrease as the temperature increases, many lubricants which work at lower temperatures are not thick enough to work at higher temperatures and those that are thick enough at the higher temperatures have a tendency to be too thick to work at the lower temperatures.

For example, the automotive industry requires lubricants which can perform across a wide range of conditions, like those found in an engine. Automotive lubricants must reduce friction between engine components when it is started from cold (relative to engine operating temperatures) as well as when it is running (up to 200° C.). The best oils (i.e. lubricants) will not vary much in viscosity over such a temperature range and therefore will perform well throughout.

In order to better predict the range of temperatures at which a lubricant would work, the Society of Automotive Engineers established the Viscosity Index. The Viscosity Index highlights how a lubricant's viscosity changes with variations in temperature. The Viscosity Index shows the viscosity of materials at an arbitrary “low” temperature of 100° Fahrenheit (40° C.) and an arbitrary “high” temperature of 210° F. (100° C.).

After understanding the properties of lubricants over the set temperature ranges, it was discovered that adding certain types of compounds to the lubricants would make the viscosity of the lubricants more consistent through a broader temperature range. Thus, there was less of a decrease in the viscosity of the lubricant at the higher temperatures. Having a higher viscosity at the higher temperature allowed the lubricants to work better at the higher temperatures. The materials added to increase the viscosity at higher temperatures were defined as viscosity index improvers.

It has surprisingly been discovered that application of that principle also has relevance to denture adhesives. In general, denture adhesive compositions comprise a denture adhesive component (salts of AVE/MA, for example) dispersed in a water insoluble component (petrolatum, for example). During use, the moisture in the saliva penetrates through the water insoluble component and hydrates the denture adhesive component. This makes the denture adhesive component sticky to the mucosal tissue and denture surface. The amount of hydration is influenced by the amount of denture adhesive component, the amount of water insoluble vehicle, and the viscosity of the water insoluble vehicle, all three of which contribute to the overall viscosity of the denture adhesive. The viscosity of the denture adhesive composition contributes to the rate and/or amount of hydration of the denture adhesive component. Over time, excess hydration due to excess saliva and/or liquids can lead to loss of some of the adhesive, thereby weakening it. As such, a denture adhesive composition that has a higher viscosity at mouth temperature due at least in part to the water insoluble vehicle would be more resistant to hydration. Simply put, the temperature-resistance of the viscosity imparted by the viscosity index improver results in resistance to excess hydration, which in turn results in more adhesive being retained over time—leading to extended and improved performance of the denture adhesive.

Thus, the use of viscosity index improvers alone or in combination with a water insoluble component will improve the hydration characteristics of a denture adhesive and thus provide an improved hold. The temperature range most relevant for denture adhesives is from room temperature (about 25° C.) which deals with the viscosity of the denture adhesive in the dispenser (outer package or inner dispensing package, for example) to about 40° C. which deals with the viscosity of the denture adhesive in the mouth. While the temperatures in the mouth can reach upward of 60° C. when drinking a hot beverage, looking at the behavior at 40° C. tends to be a good predictor of having increased beneficial properties at 60° C. as well. Thus, viscosity index improvers relevant for denture adhesives will make the viscosity more stable over the range of functional temperatures (i.e. about 25° C. to about 60° C.).

In light of the above, in one embodiment the denture adhesive article comprises a homogeneous mixture of the denture adhesive component and the viscosity index improver. In another embodiment, the denture adhesive article comprises a uniform mixture of the adhesive component dispersed within the viscosity index improver.

In one embodiment, the denture adhesive composition is in the form of an article.

Regardless of the form of dispensing the article, including but not limited to pre-dosed ready to use articles and/or articles which are dispensed from, for example, a tube, articles are substantially solid prior to use and can be picked up manually. Denture adhesive articles that can be dispensed from a tube can be identified as articles by the following method:

Please read all steps before starting test.

1. Fill the product into a tube with a 0.16″ diameter nozzle.

2. Extrude a 1″ long strip of the product onto a denture tile (1.5″×1.5″ square tile made from denture-plastic) taking care to hold the nozzle about ⅛″ above the denture tile. Do not touch the nozzle to the denture tile while extruding the product.

3. After about 1″ of product has been extruded, hold the nozzle about ⅛″ above the denture tile and use a spatula to cut the strip against the nozzle. Do not touch or smear the nozzle against the denture tile while cutting the strip.

4. Use the thumb and forefinger to hold the middle of the strip and pick it up vertically off the denture tile. Do not use a wiping motion of the fingers against the denture tile.

5. The composition is an article if it can be picked up in substantially one piece.

Substantially in one piece means, as used herein, that from about 75%, 80%, 85%, 90% to about 100%, 90%, 85%, 80%, 75%, 70% and/or any combination thereof of the denture adhesive composition remains in one piece when manually picked up from the oral surface.

Some denture adhesive articles are pre-dosed and/or ready to use. A user may be able to identify these items visually as a denture adhesive article, as they are often in the form of a strip contained within a package. However, if not evident that these denture adhesive products are articles, these denture adhesive articles can be identified as articles by the following method:

1. Shape the composition into a sheet about 0.67 mm thick×about 8 mm wide×about 44 mm long.

2. Place sheet on a denture tile.

3. Use fingers to pick up sheet.

4. The composition is an article if it can be picked up in substantially one piece.

In addition to taking many forms, the denture adhesive composition can also take many shapes. These shapes include, for example, symmetrical and asymmetrical concave shapes, lines, dots, dashes, flat, rounded, etc.

The denture adhesive composition also has many properties. In one embodiment, the composition has a property selected from the group consisting of: bioerodible, non-aqueous, and mixtures thereof.

In one embodiment, the denture adhesive composition is substantially free of: water, polyethylene oxide, Eudragit, cellulose, acrylic, polydimethylsiloxene, room temperature vulcanizing compositions, two-part compositions, epoxies, water soluble thermoplastic components, non-crystalline thermoplastic components, hydrophobic acetate, acrylic ester, polyvinyl alcohol, polyethylmethylacrylate (PEMA), polymethylmethylacrylate (PMMA), polybutylmethylacrylate (PBMA), and/or combinations thereof.

In some embodiments the composition of the present invention must be cleaned off of the denture after use, cannot be easily removed from the denture once applied, and/or easily separated from the denture after it has been used in the mouth. In some embodiments the composition of the present invention erodes within three days and is not capable of being used for a period of for example one week to a few weeks. In some embodiments the composition of the present invention is not a self-supporting and shape-retaining layer in the form of a sheet, and/or, a liner that can be used over one to three days. In some embodiments the composition of the present invention does not cure and set, is not self-supporting, does not retain its shape, is not in the form of a sheet, is not rubber-like, is not flexible, and/or is not a liner.

### Denture Adhesive Articles

The articles of the present invention have numerous benefits. In one embodiment the articles herein minimize or avoid the problem of premature sticking during application of the article to the denture. That is, with some prior art denture adhesive articles, before the article can be properly positioned over a target surface on the denture, inadvertent contact of the article with the denture may cause premature sticking at one or more locations on the denture. This may inhibit proper positioning of the article. Premature sticking may also cause contamination or degradation of the article prior to final positioning on the denture.

In one embodiment the term “dry tack” as used herein means that present articles exhibit minimal and/or no adhesive or cling properties in the dry state until activated by pressure applied by a user. In one embodiment this characteristic permits the present articles to be stored and dispensed in any desired mode without encountering the difficulties of premature clinging or adhering to themselves, and without the need for separate release sheets, liners, spacers, or the like. At the same time, in one embodiment when pressure activated at the desired location and at the desired time, the articles can, in the dry state, exhibit sufficient adhesive properties to form a bond to most plastic surfaces including a denture surface, this bond being sufficiently strong to survive handling of the denture without bond failure. Therefore, in one embodiment the articles herein, in the dry state, adhere to a target denture surface only when pressed thereagainst, thereby minimizing or avoiding this problem of inadvertent adherence during positioning on the denture surface. In one embodiment then, the articles herein do not have to be moistened or wet prior to application to the denture, thus providing a simple and easy way to apply an article to the denture.

In one embodiment the term “dry tack” as used herein means that present articles exhibit minimal and/or no adhesive or cling properties until activated by pressure applied by the user after the article has been warmed by the hands of the user, potentially during the course of application of the article to the denture surface.

In another embodiment the articles herein are non-tacky to the touch prior to application to the denture.

In another embodiment the term “dry tack” as used herein means articles herein in a dry and un-wetted state, are capable of immediate bonding by surface attachment to a dry plastic, polymethyl methacrylate, and/or other denture prothesis substrate, upon subjecting the article to pressure. In one embodiment the dry article, develops bonding by surface attachment to a dry denture prosthesis substrate upon the application of finger pressure whereby the article remains bonded under its own weight, and the article herein will not remain bonded to this dry substrate under its own weight without using finger pressure to apply the article to the substrate. In one embodiment the force or pressure may be generated by one or more fingers. This force or finger pressure, in one embodiment, may be applied for 1-10 seconds or longer. In another embodiment the bonding of the article to the substrate is maintained from about 10 seconds to about 3 minutes or longer, in another embodiment from about 30 seconds to about 1 minute or longer.

In one embodiment the dry tack of the article is from about 0.025, 0.1, 1, 10, 100, 1000 gram force/square centimeter to about 10, 100, 1000, 10,000, 50,000, 100,000 gram force/square centimeter and any combination thereof.

In one embodiment the dry tack of an article that can be repositioned is from about 0.025 grams/force square centimeter to about 0.30 grams/force square centimeter, and in another embodiment from about 0.025 gram force/square centimeter to about 0.25 gram force/square centimeter.

It is reported that the room temperature modulus of any tacky adhesive is less than 3×10⁶ dynes/cm² when measured at a frequency of 1 Hz. This finding is a criterion for tack and has been given the name “Dahlquist criterion for tack.” (Adhesion and Adhesives Technology, by Alphonsus Pocius, 2^{nd} Edition, 2002 Carl Hanser Verlag, Munich).

In one embodiment of the current invention, the article has a modulus greater than the ‘Dahlquist criterion for tack’ of about 3×10⁶ dynes/cm². In another embodiment, the article has a shear storage modulus G′ (measured in dynes/cm² at a frequency of about 1 Hz at about 25 C) greater than about 5×10⁶; in another embodiment greater than about 1×10⁷; in another embodiment greater than about 5×10⁷; and in another embodiment greater than about 8×10⁷.

In one embodiment the article has a shear storage modulus G′ (measured in dynes/cm² at a frequency of about 1 Hz at about 25 C) from about 1×10⁶, 3×10⁶, 5×10⁶, 1×10⁷, 5×10⁷, and 8×10⁷ to about 5×10⁸, 5×10⁷, 1×10⁸, 5×10⁹, 1×10⁹, and 1×10¹⁰ and/or any combination thereof.

In one embodiment the article has a flexural stiffness of less than about 10 grams/cm, in another embodiment less that about 5 grams/cm, in another embodiment less that about 3 grams/cm, in another embodiment less than about 2 grams/cm and in yet another embodiment from about 0.1, 0.5, 1, to about 2, 3, 5, 10 grams/cm, in any combination, flexural stiffness as measured on a Handle-O-Meter, model #211-300, available from Thwing-Albert Instrument Company of Philadelphia, Pa. as per test method ASTM D2923-95.

In one embodiment the articles herein have a normalized dislodgement force of from about 1100 to about 12,000 grams per sq·cm, in another embodiment from about 1300 to about 10,000 grams per sq·cm, in another embodiment from about 1200 to about 5000 grams per sq·cm, in another embodiment from about 1400 to about 5000 grams per sq·cm, in another embodiment from about 1300 to about 2500 grams per sq·cm, in another embodiment from about 1750 to about 2500 grams per sq·com. In another embodiment, the normalized dislodgement force is from about 1100, about 1200, about 1300, about 1400, about 1500, about 1750 grams per sq·cm. to about 12,000, about 10,000, about 7500, about 5000, about 2500, about 2250 grams per sq·cm, and/or any combination thereof. In one embodiment the dislodgement force ratio is from about 1.1, 1.2, 1.3, 1.4, 1.5, 1.6, 1.7, 1.8, 1.9, 2.0 to about 1.3, 1.4, 1.5, 1.6, 1.7, 1.8, 1.9, 2.0, 3, 4, 5, 6, 8, 10, and/or any combination thereof. In one embodiment the dislodgement force ratio is from about 1.1 to about 10, from about 1.1 to about 8, from about 1.3 to about 4, and/or from about 1.3 to about 2.5.

The articles herein are preformed, meaning they are pre-shaped, and ready to use. The articles are substantially solid prior to use, and can be picked up manually, in substantially one piece, and positioned on the denture. The denture adhesive articles may be in the form of a strip contained within a package. In one embodiment the articles are capable of being picked up manually, and positioned on the denture, resulting in little or no residue on the fingers. In another embodiment the articles comprise a single layer. In yet another embodiment the articles are laminates, composites, and/or comprise two or more layers.

The denture adhesive articles may also be identified by the ooze method (as defined herein). A denture adhesive article may have a normalized ooze amount of from about 0, 3, 5, 10, 15, 20, 25% of the total article to about 30, 25, 20, 15, 10, 5, 3% of the total article and/or any combination thereof and/or the ooze ratio is from about 0, 00001, 0.001, 0.01, 0.1, 0.2, 0.25, 0.3, to about 0.1, 0.2, 0.25, 0.3, 0.4, 0.5 and/or any combination thereof.

In one embodiment the article herein comprises ingredients of natural origin.

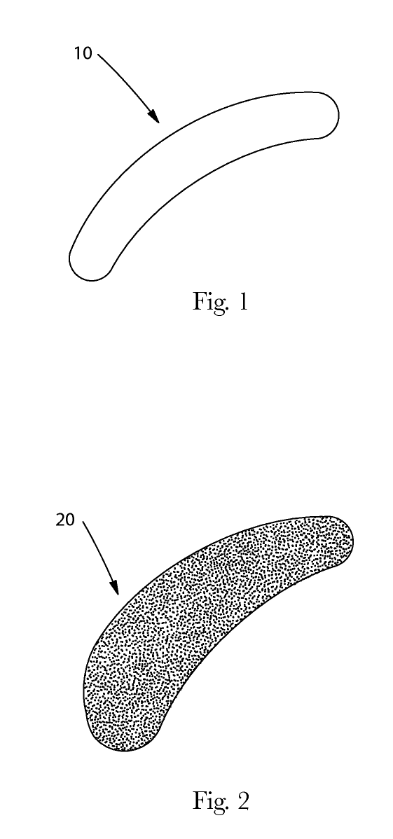

The denture adhesive article can have a variety of shapes and sizes including but not limited to a concave shape which is either symmetrical or asymmetrical. FIG. 1 shows a symmetrical concave shaped denture adhesive article 10. FIG. 2 shows an asymmetrical concave shaped denture adhesive article 20, in particular FIG. 2 shows a kidney shaped denture adhesive article 20.

In some embodiments the articles of the present invention are bio-erodible, non-aqueous, flexible, and/or thermoplastic. In some embodiments if the article contains polyethylene oxide, then the viscosity index improver and/or the optional water insoluble component is thermoplastic. In some embodiments if the article contains polyethylene oxide, the article may not include a fibrous paper web or paper laminate. In some embodiments, the article is substantially free of polyethylene oxide. In one embodiment the article herein is substantially free of honey mixed with alcohol. In another embodiment the article is substantially free of polyvinyl acetate resin in ethyl alcohol. In some embodiments the article is substantially free of polyvinyl acetate.

### Denture Adhesive Component

The present invention comprises a safe and effective adhesive amount of a denture adhesive component, generally at a level of from about 5% to about 99% by weight of the denture adhesive article. In other embodiments, the denture adhesive component is in the range from about 5%, 10%, 15%, 20%, 25%, 30%, 35%, 40%, 45%, 50%, 55%, or 60% to about 20%, 30%, 40%, 50%, 60%, 70%, 80%, 90%, or 99%, or any combination thereof. In other embodiments, the articles of the present invention comprise from at least 20%, by weight, and in another embodiment at least 30%, by weight, of the article, of a denture adhesive component.

In general, denture adhesive components are hydrophilic particles that become sticky when activated by moisture or are hydrophilic liquids. For those that activate with moisture, moisture can be present, for example, in the denture adhesive composition itself as well as in the oral cavity of the user. In varying embodiments, the denture adhesive components herein are mucoadhesive, hydrophilic, water soluble, have the property of swelling upon exposure to moisture, form a mucilaginous mass when combined with moisture, or any combination thereof. In a further embodiment the denture adhesive component is selected from the group consisting of: glycerin, poloxamer, Sorbitol, polyox, carbomer, polyacrylamides, poly peptides, natural gums; synthetic polymeric gums; AVE/MA; AVE/MA/IB; copolymers of maleic acid or anhydride and ethylene, styrene, and/or isobutylene, polyacrylic acid and/or polyacrylates thereof; polyitaconic acid, mucoadhesive polymers; water-soluble hydrophilic colloids; saccharide; cellulose; their derivatives, and combinations thereof. Examples of such materials include karaya gum, guar gum, gelatin, algin, sodium alginate, tragacanth, chitosan, acrylamide polymers, carboxypolymethylene, polyvinyl alcohol, polyamines, polyquarternary compounds, polyvinylpyrrolidone, polyvinylpyrrolidone copolymers, cationic polyacrylamide polymers, salts and mixed salts of AVE/MA, salts and mixed salts of AVE/MA/IB, salts and mixed salts of AVE/MA/Styrene, salts and mixed salts of AVE/MA/Ethylene; polymeric acids, polymeric salts, and copolymers thereof; polyitaconic acid salts, polyhydroxy compounds, their derivatives, and combinations thereof.

In one embodiment the denture adhesive component is selected from the group consisting of salts of AVE/MA, mixed salts of AVE/MA, cellulose derivatives (such as methylcellulose, carboxymethylcellulose, hydroxyethylcellulose, hydroxypropylcellulose, hydroxy-propylmethylcellulose, corn starch, and combinations thereof), polyethylene glycol, karaya gum, sodium alginate, chitosan, and combinations thereof. In yet another embodiment, the adhesive component is selected from the group consisting of mixed salts of AVE/MA, cellulose derivatives, and combinations thereof.

In another embodiment, the denture adhesive component is selected from the group consisting of: cellulose, cellulose derivatives, starch, starch derivatives, saccharide, saccharide derivatives, polyethylene oxides, polyethylene glycols, polyvinyl alcohols, carrageenan, alginates, karaya gums, xanthan gums, guar gums, gelatins, algins, tragacanth, chitosan, acrylamide polymers, carboxypolymethylenes, polyamines, poly quaternary compounds, polyvinylpyrrolidone, AVE/MA, salts of AVE/MA, mixed salts of AVE/MA, polymeric acids, polymeric salts, polyhydroxy compounds, and combinations thereof.

In one embodiment, the adhesive component is a salt of a polymer of AVE/MA. In another embodiment the adhesive component comprises a mixed salt of a polymer of AVE/MA. In a further embodiment, the AVE/MA copolymer contains a cationic salt function comprising a cation selected from the group consisting of: Group IA and Group IIA cations of the periodic table, yttrium, titanium, zirconium, vanadium, chromium, manganese, iron, nickel, copper, zinc, boron, aluminum, and combinations thereof. In another embodiment, the adhesive component is a mixed salt of an AVE/MA copolymer containing a cationic salt function comprising a cation selected from the group consisting of strontium, zinc, iron, boron, aluminum, vanadium, chromium, manganese, nickel, copper, yttrium, titanium, magnesium, calcium, sodium, and combinations thereof. In yet another embodiment the cation is selected from the group consisting of strontium, zinc, iron, magnesium, calcium, sodium, and combinations thereof. In one embodiment, the adhesive component comprises a calcium and zinc mixed salt of an AVE/MA copolymer. In another embodiment, the denture adhesive component comprises AVE/MA, salts of AVE/MA, mixed salts of AVE/MA, sodium carboxymethylcellulose, or combinations thereof. In another embodiment, the denture adhesive component is a combination of a mixed salt of AVE/MA and carboxymethylcellulose.

In further embodiments, the denture adhesive article comprises an additional adhesive component. In one embodiment, the additional adhesive component is present at the same levels and is selected from those listed for the adhesive component. In one embodiment, the additional adhesive component comprises a cellulose derivative. In a further embodiment, the cellulose derivative comprises sodium carboxymethylcellulose. In multiple embodiments, the additional adhesive component is present from about 5, 10, 15, 20% to about 30, 35, 40, 45, 50, 60%, or any combination thereof.

In one embodiment the denture adhesive component is not thermoplastic and/or comprises only low levels of water-soluble thermoplastic polymers, from about 0.01% to about 5% of water soluble thermoplastic polymers such as polyethylene oxide, hydroxypropyl cellulose, hydroxypropylmethylcellulose, and or polyethylene glycol; in another embodiment from about 0.01% to about 1% of water soluble thermoplastic polymer, or is essentially free of water soluble thermoplastic polymers.

In some embodiments when the article comprises three layers and has a denture adhesive component selected from the group consisting of sodium carboxymethylcellulose, polyethylene oxide, polyvinylalcohol, and mixtures thereof, then at least one outside layer comprises a water insoluble thermoplastic component and/or a viscosity index improver; and/or at least one layer comprises an additional denture adhesive component selected from the group comprising AVE/MA, salts of AVE/MA, mixed salts of AVE/MA, and mixtures thereof.

### Alkyl Vinyl Ether-Maleic Copolymer

In one embodiment of the invention the denture adhesive component is AVE/MA or salts of AVE/MA. The alkyl vinyl ether-maleic acid co-polymer comprises or consists essentially of the repeated structural unit:

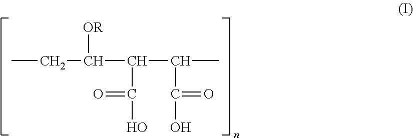

wherein R represents an alkyl radical, in one embodiment a C₁ to C₅ alkyl radical, n is an integer greater than one representing the number of repeated occurrences of the structural unit in a molecule of the polymer.

In one embodiment, the adhesive component is AVE/MA and salts thereof, preferably mixed salts of AVE/MA, wherein the copolymer contains a cationic salt function comprising a cation selected from the group consisting of Group IA and Group 2A cations of the periodic table, yttrium, titanium, zirconium, vanadium, chromium, manganese, iron, nickel, copper, zinc, boron, aluminum, and mixtures thereof. In another embodiment, the adhesive component is a mixed salt of AVE/MA containing a cationic salt function comprising a cation selected from the group consisting of strontium, zinc, iron, boron, aluminum, vanadium, chromium, manganese, nickel, copper, yttrium, titanium, magnesium, calcium, sodium, and mixtures thereof, and in yet another embodiment the cation is selected from the group consisting of strontium, zinc, iron, magnesium, calcium, sodium, and mixtures thereof.

AVE/MA contains, in one embodiment, a cationic salt function comprising from about 5% to about 50%, in another embodiment, from about 10% to about 40%, in yet another embodiment, from about 10% to about 35% (of the total initial carboxyl groups reacted) zinc cations. These zinc cations can be mixed with other cations selected from the group consisting of: from about 5% to about 65%, preferably from about 10% to about 60%, strontium cations, from about 0.001% to about 2.5%, preferably from about 0.01% to about 2% of iron, boron, aluminum, vanadium, chromium, manganese, nickel, copper, yttrium, and/or titanium cations, from about 5% to about 65%, preferably from about 15% to about 50% of calcium and/or magnesium cations and/or sodium cations.

AVE/MA and salts thereof, are also described in U.S. Pat. Nos. 5,073,604 to Holeva et al., issued Dec. 17, 1991; 5,525,652, issued Jun. 11, 1996, Clarke et al.; 6,025,411, issued Feb. 15, 2000, Wong et. al.; 4,758,630, issued Jul. 19, 1988, Shah et al.; 5,304,616, issued Apr. 19, 1994, Rajaiah et al.; 5,424,058, issued Jun. 13, 1995, Rajaiah; 5,424,058, issued Jun. 13, 1995, Rajaiah et al.; 4,758,630, issued Jul. 19, 1988, Shah et al.; 5,830,933, issued Nov. 3, 1998, Synodis et al.; 2,047,398, issued Jul. 14, 1936, Voss et al.; 3,003,988, issued Oct. 10, 1961, Germann et al.; 5,880,172, Rajaiah et al., issued Mar. 9, 1999; 5,900,470, Prosise et al., issued May 4, 1999; 5,037,924, Tazi et al., issued Aug. 6, 1991; 5,082,913, Tazi et al, issued Jan. 21, 1992; all of which are incorporated herein by reference in their entirety. Salts of AVE/MA are also described in P&G copending applications U.S. Pat. Nos. 6,355,706 to Rajaiah, et al., issued Mar. 12, 2002; 6,617,374 to Rajaiah, et al., issued Sep. 9, 2003.

In one embodiment the free acid level of the salts of the AVE/MA or AVE/MA/IB is at least about 36%, in another embodiment is from about 36% to about 60%, and even in another embodiment is from about 40% to about 55%, of the total initial carboxyl groups of the copolymer.

In one embodiment the specific viscosity of the starting copolymer acid or copolymer anhydride is from about 1.2 to about 14, when preferably measured in a 1% weight/volume solution in MEK (methyl ethyl ketone) at 25° C. Other methods and solvents can be used to measure the specific viscosity such as a 1% weight/volume solution in DMF (dimethyl formamide) at 25° C. and a 1% weight/volume solution in 2-butanone at 25° C.

Suitable AVE/MA copolymers may be prepared by well-known methods of the prior art; see, for example, U.S. Pat. No. 2,782,182, and U.S. Pat. No. 2,047,398.

Methods of making mixed salts of AVE/MA polymers are further disclosed in U.S. Pat. Nos. 5,073,604, Holeva et al., issued Dec. 17, 1991 and 5,872,161, Liang et al., issued Feb. 16, 1999.

### Water Insoluble Component

In some embodiments, the present article comprises a safe and effective amount of a water insoluble component (wic). In one embodiment this component is present by weight of the article at an amount from about 2, 5, 10, 20, 25, 30, 35% to about 45, 50, 60, 70, 90%, or any combination thereof. In additional embodiments the water insoluble component is present at an amount from about 20% to about 70%, from about 25% to about 60%, or from about 35% to about 60% by weight of the article. In yet another embodiment the water insoluble component is substantially non-swellable in water. In some embodiments, the non-swellable water insoluble component swells less than about 10%, 5%, 2%, or 1% in water.

In one embodiment, the water insoluble component comprises a liquid, gel, or mixtures thereof. In one embodiment, the water insoluble component is selected from the group consisting of: natural wax, synthetic wax, petrolatum, polyvinyl acetate, natural oils, synthetic oils, fats, silicone, silicone derivatives, dimethicone, silicone resins, hydrocarbons, hydrocarbon derivatives, essential oils, caprilic/capric triglycerides, polybutene, oleic acid, stearic acid, and combinations thereof. In a further embodiment, the water insoluble component comprises petrolatum, polyvinyl acetate, natural oils, synthetic oils, fats, silicone, silicone derivatives, dimethicone, silicone resins, hydrocarbons, hydrocarbon derivatives, polybutene, oleic acid, stearic acid, essential oils, caprilic/capric triglycerides, or combinations thereof.

Examples of natural oils include, but are not limited to, vegetable oils (ex. corn oil), soy bean oils, cottonseed oils, palm oils, coconut oils, mineral oils, animal oils (ex. fish oils), etc. Examples of synthetic oils include, but are not limited to, silicone oils, etc. In one embodiment, the water insoluble component comprises a natural oil. In other embodiments, the water insoluble component may comprise mineral jelly, for example, mineral jellies numbers 4, 5, 10, 15, or 20 from Calumet Specialty Products.

In a further embodiment, the natural oil comprises mineral oil. In one embodiment, mineral oil is present in the article at an amount from about 30% to about 50% and in another embodiment, from about 35% to about 45%. In some embodiments, the mineral oil may be white, light, or technical. Light mineral oil may be, for example, Drakeol 5, 10, 13, or 15. White mineral oil may be, for example, Drakeol 19, 21, 34, 35, or 600.

In some embodiments, the water insoluble component is substantially free of petrolatum. In another embodiment, the water insoluble component further comprises petrolatum.

In some embodiments, the water insoluble component is a wax. Waxes are generally made up of various substances including hydrocarbons (normal or branched alkanes and alkenes), ketones, diketones, primary and secondary alcohols, aldehydes, sterol esters, alkanoic acids, terpenes (squalene) and monoesters (wax esters). Different types of waxes include animal and insect waxes (beeswax, Chinese wax, shellac wax, spermaceti, lanolin), vegetable waxes (bayberry wax, candelilla wax, carnauba wax, castor wax, esparto wax, Japan wax, jojoba oil, ouricury wax, rice bran wax), mineral waxes (cresin waxes, montan wax, ozocerite, peat waxes), petroleum waxes (paraffin wax or microcrystalline wax), and synthetic waxes (polyethylene waxes, Fischer-Tropsch waxes, chemically modified waxes, substituted amide waxes, polymerized α-olefins).

In one embodiment the water insoluble component is a natural or synthetic wax. In a further embodiment, the natural wax is selected from the group consisting of: animal wax, vegetable wax, mineral wax, and combinations thereof. In another embodiment, the animal wax includes beeswax, lanolin, shellac wax, Chinese wax, and combinations thereof. In another embodiment, the vegetable waxes include carnauba, candelilla, bayberry, sugar cane, and combinations thereof; and mineral waxes include fossil or earth waxes (ozocerite, ceresin, montan), and petroleum waxes such as paraffin and microcrystalline wax, and combinations thereof. In one embodiment the waxes herein are natural waxes selected from the group consisting of beeswax, candelilla, candela, carnauba, paraffin, and combinations thereof. In varying embodiments, wax can be present in an amount from about 1, 2, 5, 8, 10, 15, 20, 30, or 40% to about 5, 10, 20, 30, 40, 50, 60, 70, or 80%, or any combination thereof.

In another embodiment, the natural wax comprises paraffin wax. A paraffin wax useful herein generally can have a melting point range of from about 65° C. to about 80° C. and, in another embodiment, from about 70° C. to about 75° C. In another embodiment, a microcrystalline wax useful herein can have a melting point of from about 65° C. to about 90° C., and, in another embodiment from about 80° C. to about 90° C. In one embodiment, a beeswax useful herein can have a melting point of from about 62° C. to about 65° C. and a flash point of 242° C. In another embodiment, a candelilla wax useful herein can have a melting point of from about 68° C. to about 72° C. In an additional embodiment, a carnauba wax useful herein can have a melting point of from about 83° C. to about 86° C. In one embodiment, a Fischer-Tropsch wax useful herein can have a melting point of about 95° C. to about 120° C. Synthetic grades of beeswax, candelilla, and carnauba waxes are also available with similar properties as the natural grades.

In one embodiment, the water insoluble component is petrolatum. According to Hawley's Condensed Chemical Dictionary 13^{th} Edition, John Wiley & Sons, 1997, petrolatum is a “mixture of hydrocarbons derived by distillation of paraffin-base petroleum fractions”; and according to The United States Pharmacopia 2005, petrolatum is a “purified mixture of semisolid hydrocarbons obtained from petroleum”. This is also referred to as “natural petrolatum”. Petrolatum is stated to have a melting range between 38° C. and 60° C. according to The United States Pharmacopia 2005, and 38-54 C according to The Merck Index, 10^{th} Edition, 1983. Petrolatums are available in a variety of grades with the “Cone Penetration Values” ranging from 180 to about 245 measured using ASTM D-937 according to the Sonneborn Inc product brochure.

In one embodiment, the water insoluble component has a melting point greater than about 60° C. In some embodiments, the water insoluble thermoplastic component has a melting point from about 35° C., 40° C., 45° C., 50° C., 55° C., 60° C., 65° C., 70° C., 75° C., 80° C., 85° C., 90° C., 95° C., 100° C., to about 110° C., 120° C., 150° C., 175° C., 200° C. and/or any combination thereof to form a range, starting point, and/or end point. In another embodiment, the composition is substantially free of a water insoluble thermoplastic component with a melting point above about 75° C.

### Viscosity Index Improvers

As discussed previously, a viscosity index improver (vii) makes the viscosity of denture adhesives more stable over a range of functional temperatures (i.e. about 25° C. to about 60° C.). It is believed that another mechanism also contributes to the improved properties of denture adhesives comprising viscosity index improvers. Without being limited by theory, it is believed at least some improved properties arise when at least some of the particles of an adhesive component are at least partially coated or surrounded by a viscosity index improver. In fact, it has been surprisingly discovered that in at least some embodiments of the present invention, a viscosity index improver, microcrystalline wax for example, can at least partially coat the particles of an adhesive component. This is especially seen when the denture adhesive article is made by heating up to or beyond the softening point of the viscosity index improver and then cooled to room temperature. Substantially more of this coating or partial coating can be visible under a polarized microscope than for a composition made utilizing a “standard” blend of mineral oil and petrolatum. In some embodiments, the viscosity index improver can coat the particles of the adhesive component by solidifying or crystallizing within the pores and/or crevices of particles of the adhesive component. In some embodiments, this coating or partial coating or pore-clogging by the viscosity index improver can then regulate the uptake of saliva into the particles of denture adhesive, thus potentially optimizing the activation of the denture adhesive.

In some instances, the coating/surrounding of the adhesive component by the viscosity index improver functions as a physical barrier to protect the adhesive particles, for example, from being washed out due to incomplete hydration, excess hydration (from saliva or drinks), change in mouth temperature (for example, due to drinking a hot beverage like coffee), and/or chewing. This can also lead to a better utilization and optimization of the adhesive component which leads to a better performance. The increase in performance can lead to the ability to use less of the product to get the same or better hold as previous products.

In some embodiments of the present invention, the water insoluble component can offer an increased resistance to moisture penetration. In some embodiments, this could be due to the coating mechanism and/or pore-clogging mechanism described above and/or simply due to the hydrophobicity of the water insoluble component surrounding the particles of denture adhesive. In some embodiments, the water-insoluble component can offer an increased resistance to moisture penetration simply due to a higher resistance to diffusion of moisture into and through the water insoluble liquid and/or water insoluble thermoplastic component surrounding the particles of denture adhesive.

In some embodiments of the present invention, a viscosity index improver component such as microcrystalline wax can crystallize out when heated and subsequently cooled, even in the presence of an excess amount of a water insoluble liquid like mineral oil. It may be reasonable to expect that petrolatum would crystallize out when heated and subsequently cooled when blended with mineral oil in about a 1:1 ratio or more petrolatum-rich ratio. In some embodiments of the present invention a crystalline viscosity index improver component such as microcrystalline wax crystallizes out when heated and subsequently cooled, even when blended with mineral oil in an ultra-lean ratio of about 1:8.

In further embodiments, these crystals of a viscosity index improver component such as microcrystalline wax are “temperature-resistant” in that they do not melt away as quickly as the crystals from petrolatum when the composition is heated to about mouth temperature or above, such as when the consumer drinks hot liquids. In some embodiments, upon heating a blend of water insoluble liquids such as mineral oil and a viscosity index improver component like microcrystalline wax, more crystals are visible at or above body temperature when viewed under a polarized light microscope, than say a blend of mineral oil and petrolatum.

It is believed that the presence of these crystals or coating can interrupt or inhibit the formation of a continuous gel-blocking layer, which can then lead to superior hydration. It is also believed that the crystals or coating on the denture adhesive particles can regulate the particle-particle sticking or interaction, thereby leading to potentially less gel-blocking and superior hydration.

In some embodiments, a blend of water insoluble liquids such as mineral oil and a viscosity index improver component like microcrystalline wax has a higher level and/or intensity of light scattering than a blend of mineral oil and petrolatum, as measured using Phase Technology Instrumentation Model No. NK60-KPA.

Some embodiments comprising a viscosity index improver component such as microcrystalline wax may have a viscosity that is substantially higher than a composition utilizing a blend of mineral oil and petrolatum, when measured at about mouth temperature. It is believed that the heat-resistant crystals are associated with the resistance to viscosity degradation due to heat. This increased viscosity at or above mouth temperature can in turn lead to a more cohesive composition during use in the mouth, especially when drinking hot liquids. This increased cohesiveness can in turn contribute directly to superior performance and/or less adhesive washing away.

Some embodiments of the present invention comprising a viscosity index improver component such as microcrystalline wax blended into mineral oil may deliver substantially superior consumer-noticeable hold performance, as compared to a blend of petrolatum and mineral oil, even when all the other components, including the denture adhesive components, are maintained to be the same for both compositions. It is believed that it is the unique properties of microcrystalline wax utilized in some embodiments of the present invention (for example, molecular structure, molecular weight, branching, and/or the manner in which it interacts physically or chemically with the denture adhesive components) that leads to superior performance compared to traditional denture adhesive creams made utilizing petrolatum. But some embodiments of the present invention may possess superior performance completely independent of the mechanisms described above.

One way to determine whether a material would work as a viscosity index improver in a denture adhesive article of the present invention is to look at the instant viscosity ratio. The instant viscosity ratio is the ratio of the viscosities at room temperature (25° C.) and at an elevated temperature (40° C.) for a particular material. Denture adhesives comprising a viscosity index improver tend to have a viscosity that is higher at elevated temperatures than those same compositions without a viscosity index improver. This is important because the denture adhesive is placed (along with the denture) into the mouth of a user which has a temperature generally higher than that of room temperature. Additionally, the temperature of a user's mouth can also be increased when ingesting hot beverages. The ability to maintain a higher viscosity at these higher temperatures contributes to better hold and less loss of the denture adhesive composition during use.

The instant viscosity ratio can be measured as outlined further below. In one embodiment, the instant viscosity ratio of the prototype sample or a material comprising a viscosity index improver is greater than about 0.25. In another embodiment, the instant viscosity ratio is from about 0.25 to about 1.0. In additional embodiments, the instant viscosity ratio is from about 0.25, 0.3, 0.4, 0.6, 0.7 to about 0.3, 0.4, 0.5, 0.8, 1.0, or any combination thereof. In a further embodiment, the instant viscosity ratio is from about 0.3 to about 0.8. In other embodiments, the instant viscosity ratio is from about 0.3 to about 0.6 or from about 0.3 to about 0.5. In one embodiment, viscosity index improvers can be considered to be a subset of the water insoluble components for which the instant viscosity ratio of a composition comprising the material is greater than about 0.25.

The following describes two samples, a reference sample (RS) and a prototype sample (PS). The reference sample is considered the standard and is made using the standard water insoluble components, which would not include a viscosity index improver, while the prototype sample is made using a viscosity index improver. A general formula is given for a RS and a PS, then formulas of a specific RS and a specific PS are given, along with their instant viscosity ratios for comparison.

Procedure to Prepare a Reference Sample (RS) and a Prototype Sample (PS)

### Materials

1. Standard Denture Adhesive Components and Excipient Powders (to prepare samples of both the RS and PS):
   i. Ca(47.5)/Zn(17.5) MVE/MA (Methyl Vinyl Ether/Maleic Acid) mixed partial salt (33%)
   ii. Sodium Carboxymethylcellulose (20%)
   iii. Colloidal Silicon Dioxide (1.14%)
2. Water Insoluble Components (WIC) and Viscosity Index Improver
   iv. To prepare a sample of the RS using standard WIC:
      Mineral Oil (Drakeol 35 from Penreco, or Kaydol from Sonneborn) (23.95%)+White Petrolatum (“Snow” from Penreco) (21.91%)
   OR
   To prepare a sample of the PS using the prototype viscosity index improver and WIC:
      Mineral Oil (Drakeol 35 from Penreco, or Kaydol from Sonneborn) (40.812%)+Prototype viscosity index improver (5.048%)

### Procedure

The Reference Sample and Prototype Sample are both prepared using the following procedure:

Connect a mixer with wall-scraper blades (Unimix from Haagen and Rinau) and hot water jacket to a water bath and a vacuum pump. Set the water bath of the hot water jacket to about 95° C. Add the WIC and/or viscosity index improver ingredients to mixer vessel. If the water insoluble component and/or viscosity index improver are not liquid at room temperature, allow them to soften before turning on the agitator. Turn on the agitator to about 60 RPM; mix the WIC and/or viscosity index improver ingredient(s) until their temperature reaches about 95° C. Add the “Standard Denture Adhesive Components and Excipient Powders” via a funnel to the mixer with the vent open. Close the vent and stop mixing. Scrape off powder clumps. Re-start mixing at about 60 RPM. Pull about 24 inches Hg vacuum and mix until the batch reaches about 90° C. Reduce bath temperature to about 60° C. and continue mixing under vacuum until the batch reaches about 65° C. Stop mixing, turn off the pump, slowly open the vent, release the vacuum, and raise the lid. Fill the sample into a suitable container, such as a foil tube of about 1.4 oz in capacity. Allow samples to equilibrate for about one week. Just prior to testing, squeeze out and discard approximately the first 2 grams from the tube(s).

Whenever possible, the RS and PS are made with the same denture adhesive components and excipient powders at the same levels and with the same manufacturing procedure. This is done to provide a standard matrix to test the differences between a variety of viscosity index improvers by keeping all other variables including the denture adhesive components and sample preparation procedure the same. Among other properties imparted by the standard denture adhesive components, they also provide a standard driving force for the saliva and moisture to penetrate through the denture adhesive composition, and also provide a standard matrix to test the effect of a variety of viscosity index improvers.

If it is necessary to accommodate any property of the Prototype viscosity index improver or viscosity index improver/water insoluble component combination that is not accommodated by the process detailed above (for example if it softens only at temperatures greater than 95° C.), the processing temperature profile can be modified as needed. Similarly, if the above blend of standard denture adhesive components is not suitable, then, just a single denture adhesive component, for example, sodium carboxymethylcellulose at 53%, can be used instead of the blend with Ca/Zn MVE/MA salt. Additionally, if the above testing formulation gives a PS which is too thick to test for the instant viscosity ratio as described below, then the sample may need to be diluted with additional water insoluble component like mineral oil.

The above process tests for viscosity index improvers at a level of about 5%. It is believed that testing the prototype viscosity index improvers at 5% will help set-up a baseline, meaning that a finding of viscosity index improver properties at a level of 5% is indicative of viscosity index improver properties at high levels. That being said, a prototype viscosity index improver which is tested at 5% and is found not to have viscosity index improver properties at that level may have them at a higher percentage and should be tested at a higher level to confirm.

The above process can also be scaled up and used for general manufacturing at the temperature appropriate for the viscosity index improver and/or water insoluble component of the denture adhesive article.

The following table, Table 1, includes formulas for a particular reference sample (RS) and a particular prototype sample (PS). These are not examples of embodiments of the present invention, but are disclosed to illustrate the difference in instant viscosity ratio when a viscosity index improver is used, in this case, microcrystalline wax.

TABLE I

| | A | B |
|---|---|---|
| | RS | PS |
| | % | % |
| Ca/Zn AVE/MA Salt | 33 | 33 |
| CMC | 20 | 20 |
| Silica | 1.14 | 0.00 |
| Mineral Oil | 23.95 | 40.812 |
| Petrolatum | 21.91 | 0.00 |
| Colloidal Silicon Dioxide NF | 0.00 | 1.14 |
| Microcrystalline Wax W-835 | 0.00 | 5.048 |
| (by Witco Crompton, | | |
| Sonneborn) | | |

The instant viscosity of the PS at 25° C. is 211.4 Ps and at 40° C. is 80.7 Ps. This gives an instant viscosity ratio for the PS of 0.38. In contrast to this, the RS, made using the combination of mineral oil and petrolatum used in traditional denture adhesive creams, has an instant viscosity at 25° C. of 289.9 Ps and at 40° C. of 51.4 Ps. This gives an instant viscosity ratio for RS of 0.18. The higher instant viscosity ratio of PS shows that it is more temperature resistant than the reference/traditional water insoluble component and thus, microcrystalline wax will work as a viscosity index improver in that denture adhesive article.

Some examples of viscosity index improvers include polymethacrylates, olefin copolymers, hydrogenated styrene-diene copolymers, styrene polyesters, rubber, polyvinylchloride, nylon, fluorocarbon, polyurethane prepolymer, polyethylene, polystyrene, polypropylene, cellulosic resins, acrylic resins, microcrystalline wax, elastomers, poly(n-butyl vinyl ether), poly(styrene-co-maleic anhydride), poly(alkyl fumarate co-vinyl acetate), alkylated polystyrene, poly(t-butyl styrene), or combination thereof.

In one embodiment, the viscosity index improver is paraffin wax. The “Kirk-Othmer Encyclopedia of Chemical Technology”, 5^{th} Edition, vol. 26, page 216, hereby incorporated by reference, states that paraffin wax has the following typical properties: flash point, closed cup, 204° C.; viscosity at 98.9° C., 4.2-7.4; melting range, 46° C.-68° C.; refractive index at 98.9° C., 1.430 to 1.433; average molecular weight, 350 to 420; carbon atom per molecule, 20 to 26; and ductibility/crystallinity of solid wax, friable to crystalline, and in one embodiment, the viscosity index improver has these particular properties.

Examples of polymethacyrlates include, for example, polyacrylate-co-methacrylate, polymethacrylate-co-styrene, or combinations thereof. Examples of elastomers include, for example, hydrogenated styrene-co-butadiene, hydrogenated styrene-co-isoprene, ethylene-ethylene-propylene polymer, ethylene-propylene polymer, styrene-ethylene-ethylene-propylene-styrene polymer or combinations thereof. An example of a rubber includes hydrogenated polyisoprene. Other examples of viscosity index improvers can be found in “Chemistry and Technology of Lubricants,” R. M. Mortier and S. T. Orszulik, Chapman and Hall (2^{nd} Ed. 1997), pages 144-180, hereby incorporated by reference.

In another embodiment, the viscosity index improver is polyethylene, such as A-C 1702 or A-C 6702 made by Honeywell, with a penetration value of about 98.5 and about 90.0, respectively, under ASTM D-1321. In some embodiments, the composition and/or the water-insoluble component is substantially free of amorphous polyethylene having a molecular weight of at least about 80,000. In an additional embodiment, when the viscosity index improver consists of a polyethylene having an average molecular weight of from about 1000 to about 21,000 then the adhesive component is substantially free of a mixed partial salt of a lower alkyl vinyl ether-maleic anhydride salt of calcium and alkali cations selected from the group consisting of sodium, potassium, and quaternary ammonium cations.

In some embodiments, the viscosity index improver and/or water insoluble component is substantially free of polyethylene, propylene glycols, glycerin, water, polyethylene oxide, polyethylene glycol, Polybutene, petrolatum, Eudragit, ethanol, alcohols, polyvinylacetate, cellulosic derivatives, honey, alcohol, acrylic derivatives, silicones, silicone derivatives, polydimethylsiloxenes (PDMS), PDMS derivatives, functionalized PDMS, end-functionalized PDMS, Room Temperature Vulcanizing compositions (RTV), two-part compositions, epoxies, polyvinylacetate, plasticizers, water soluble plasticizers, water soluble thermoplastic components, water soluble liquids, and/or non-crystalline thermoplastic components, hydrophobic acetate, acrylic ester derivative, PVA, PEMA, PMMA, PBMA, and/or, Triacetin.

In one embodiment the viscosity index improver is water-insoluble, hydrophobic, and/or thermoplastic.

In another embodiment, the viscosity index improver comprises microcrystalline wax. In one embodiment, the microcrystalline wax is refined and/or substantially pure. In an additional embodiment, petrolatum does not contribute the microcrystalline wax. The “Encyclopedia of Polymer Science and Engineering”, 2^{nd} Edition, Vol. 17, page 788, hereby incorporated by reference, states that the molecular weight of microcrystalline wax ranges from 450 to 800. The “Kirk-Othmer Encyclopedia of Chemical Technology”, 5^{th} Edition, vol. 26, page 216, hereby incorporated by reference, states that microcrystalline wax has the following typical properties: flash point, closed cup, 260° C.; viscosity at 98.9° C., 10.2-25 mm²/s; melting range, 60° C.-93° C.; refractive index at 98.9° C., 1.435 to 1.445; average molecular weight, 600 to 800; carbon atom per molecule, 30 to 75; and ductibility/crystallinity of solid wax, ductile-plastic to tough-brittle, and in one embodiment, the viscosity index improver has these particular properties.

In one embodiment, the viscosity index improver comprises n-alkanes. In one embodiment, the viscosity index improver comprises n-alkanes with greater than about 30 carbon atoms.

Microcrystalline wax has been measured to have a total alkyl branching of about 12%, while petrolatum has about 43%, as measured using ¹³C-NMR.

In another embodiment, the microcrystalline wax has a melting point ranging from about 50° C. to about 100° C. In further embodiments, the microcrystalline wax has a melting point ranging from about 50° C., 55° C., 60° C., 65° C., 70° C. to about 70° C., 75° C., 80° C., 85° C., 90° C., 95° C., 100° C., or any combination thereof. In one particular embodiment, the microcrystalline wax has a melting point ranging from about 75° C. to about 85° C.

In another embodiment the microcrystalline wax is manufactured by Crompton, Sonneborn (Witco) and referred to and sold under the trademark Mutiwax®W-835. This wax has a melting point ranging from about 73.9° C. to about 79.4° C. (measured using ASTM D-127), has a penetration at 25° C. of from about 60 to about 80 (measured using ASTM D-1321), has a kinematic viscosity at 98.9° C. of from about 75 to about 90 saybolt universal seconds (measured using ASTM D-2161), has a flash point, COC (Cleveland open cup), of at least about 246° C. (measured using ASTM D-92), and has a congealing point from about 68° C. to about 77° C. (measured using ASTM D-938).

In another embodiment the microcrystalline wax is manufactured by Crompton, Sonneborn (Witco) and referred to and sold under the trademark Mutiwax®180W. This wax has a melting point ranging from about 79° C. to about 87° C. (measured using ASTM D-127), has a penetration at 25° C. of from about 15 to about 22 (measured using ASTM D-1321), has a kinematic viscosity at 98.9° C. of at least about 75 saybolt universal seconds (measured using ASTM D-2161), has a flash point, COC (Cleveland open cup), of at least about 277° C. (measured using ASTM D-92), and has a congealing point from about 75° C. to about 82° C. (measured using ASTM D-938).

In another embodiment the microcrystalline wax is manufactured by Crompton, Sonneborn (Witco) and referred to and sold under the trademark Mutiwax®W445. This wax has a melting point ranging from about 77° C. to about 82° C. (measured using ASTM D-127), has a penetration at 25° C. of from about 25 to about 35 (measured using ASTM D-1321), has a kinematic viscosity at 98.9° C. of from about 75 to about 90 saybolt universal seconds (measured using ASTM D-2161), has a flash point, COC (Cleveland open cup), of at least about 277° C. (measured using ASTM D-92), and has a congealing point from about 72° C. to about 77° C. (measured using ASTM D-938).

While microcrystalline wax and paraffin wax are both petroleum waxes, there are specific differences between them. Microcrystalline wax is a refined mixture of solid, saturated aliphatic hydrocarbons produced by de-oiling certain fractions from the petroleum refining process. In contrast to the more familiar paraffin wax which contains mostly unbranched alkanes, microcrystalline wax contains a higher percentage of isoparaffinic (branched) hydrocarbons and naphthenic hydrocarbons. It is characterized by the fineness of its crystals in contrast to the larger crystal of paraffin wax. It consists of high molecular weight saturated aliphatic hydrocarbons. It is generally darker, more viscous, denser, tackier and more elastic than paraffin waxes, and has a higher molecular weight and melting point. The elastic and adhesive characteristics of microcrystalline waxes are related to the non-straight chain components which they contain. Typical microcrystalline wax crystal structure is small and thin, making them more flexible than paraffin wax.

According to the “Encyclopedia of Polymer Science and Engineering” Volume 17 page 788, 1989 John Wiley & Sons): The molecular weights of paraffin waxes range from about 280 to 560 (C20 to C40); the molecular weights of microcrystalline wax range from 450 to 800 (C35 to C60). The amount of n-alkanes in paraffin wax usually exceeds 75% and can be as high as 100%; microcrystalline waxes are composed predominantly of iso-paraffinic and napthenic saturated hydrocarbons along with some n-alkanes.

According to Kirk-Othmer Encyclopedia of Chemical Technology, John Wiley & Sons, 2005: Paraffin Waxes have a number average molecular weight of 350-420 and carbons per molecule of 20-36; and Microcrystalline waxes have a number average molecular weight of 600-800 and carbons per molecule of 30-75. Paraffin wax is macrocrystalline, brittle, and is composed of 40-90% normal alkanes, with the remainder C18-C36 isoalkanes and cycloalkanes. A paraffin wax is a petroleum wax consisting principally of normal alkanes. Microcrystalline wax is a petroleum wax containing substantial proportions of branched and cyclic saturated hydrocarbons, in addition to normal alkanes. A classification system based on the refractive index of the wax and its congealing point as determined by ASTM D-938 has been developed. Paraffin waxes have a refractive index at 98.9 C of 1.430-1.433; and microcrystalline waxes have a refractive index at 98.9 C of 1.435-1.445. Paraffin waxes are friable to crystalline; microcrystalline waxes are ductile-plastic to tough-brittle. Paraffin wax has little affinity for oil; microcrystalline wax has great affinity for oil. Unlike paraffin wax, oil is held tightly in the crystal lattice of the microcrystalline wax, and does not migrate to the surface. Paraffin wax is stated to have a melting point of about 47-65° C., according to Hawley's Condensed Chemical Dictionary 13^{th} Edition, John Wiley & Sons, 1997, and 46-68° C., according to Kirk-Othmer Encyclopedia of Chemical Technology, John Woley & Sons, 2005. Microcrystalline wax is stated to have a melting point of about 63-88° C., according to Hawley's Condensed Chemical Dictionary 13^{th} Edition, John Wiley & Sons, and 60-93° C., according to according to Kirk-Othmer Encyclopedia of Chemical Technology, John Wiley & Sons, 2005.

In some embodiments, the water insoluble thermoplastic and/or viscosity index improver used in the present invention have a Penetration Value from about 5, 10, 15, 20, 25, 30, 35, 40, 45, 50, 55, 60 to about, 25, 30, 35, 40, 45, 50, 55, 60, 65, 70, 75, 80, 85, 90, 95, 100, 110, 120, 250, in any combination of numbers to form ranges.

In some embodiments, the water insoluble thermoplastic component and/or viscosity index improver such as microcrystalline wax has an average molecular weight higher than that of petrolatum. In some embodiments the water-insoluble component and/or viscosity index improver is higher in MW, more branched, more flexible, stronger, tougher, higher melting, and/or more crystalline than blends of mineral oil combined with petrolatum.

Low density polyethylene is stated to have a melting point of about 116° C., and high density polyethylene a melting point of about 135° C. according to Hawley's Condensed Chemical Dictionary 13^{th} Edition, John Wiley & Sons. Polyethylene grade A-C 6A, 8A, 9A have a drop point ranging from 106 to 115° C. measured using ASTM D-3954 (product label from Honeywell).

Unless otherwise noted, the term “melting point” as used herein refers to the “drop melting point”, which is the temperature at which the material becomes sufficiently fluid to drop from the thermometer used in making the determination under definite prescribed conditions in ASTM D-127; if the method in ASTM D-127 is found to be substantially not suitable to measure the melting point of certain materials, the “dropping point” as measured by ASTM D-3954 can be used instead.

Additional details about waxes, petroleum products, viscosity index improvers, other ingredients and methods pertinent to the compositions in this patent application may be found in the following references (sections pertaining to waxes, petroleum products, viscosity index improvers, other ingredients and methods are hereby incorporated by reference):
1. “Encyclopedia of Polymer Science and Engineering” all editions, John Wiley & Sons,
2. “Kirk-Othmer Encyclopedia of Chemical Technology”, all editions, John Wiley & Sons,
3. “Hawley's Condensed Chemical Dictionary”, all editions, John Wiley & Sons.
4. The United States Phamacopia, all editions.
5. The Merck Index, all editions.

In one embodiment, the viscosity index improver comprises microcrystalline wax and is present at an amount from about 10% to about 90% and in another embodiment from about 40% to about 60%.

In some embodiments, viscosity index improvers are used in an amount from about 1% to about 90%, and about 10% to about 90% in other embodiments. In another embodiment, the viscosity index improvers are used in an amount from about 40.0% to about 60.0%. In additional embodiments, the viscosity index improver is present from about 20, 30, 40, 45, 50, or 55% to about 25, 30, 40, 50, 60, 70, 80, or 90%, or any combination thereof. In one embodiment, the viscosity index improver is water soluble and/or non-swellable in water.

### Non-Adhesive Substrate

In one embodiment the present invention comprises at least one non-adhesive self supporting substrate. In one embodiment the non-adhesive self-supporting substrate is characterized by its ability to maintain strength and provide integrity for the article in the presence of water and/or saliva.

In one embodiment, the user or consumer is able to easily peel away the article in its original form, from the prosthesis after the article is used in the oral cavity as a denture adhesive.

In one embodiment the non-adhesive self-supporting substrate is a solid substrate material having a penetration value of less that about 20, in another embodiment less than about 10, and in another embodiment less than about 5, based on ASTM D1321 or ASTM D937. In one embodiment the non-adhesive self-supporting substrate is a solid substrate material having a penetration value of about 0.5, 1, 2 to about 10, 15, 18, 20, or any combination of these, based on ASTM D1321 or ASTM D937.

In one embodiment the non-adhesive self-supporting substrate is a substrate with a hardness value of greater than about 0.5, in another embodiment greater than about 1, and in another embodiment greater than about 2, under ASTM D5. In one embodiment the non-adhesive self-supporting substrate is a solid substrate material having a hardness value of about 0.5, 1, 2 to about 10, 15, 18, 20, or any combination of these, based on ASTM D5.

In one embodiment the non-adhesive self-supporting substrate may include materials such as polyester, polypropylene, nylon, rayon, cellulose acetate, non-adhesive cellulose derivatives, cellulose acetate, cloth, fibrous fleece, paper, plastic, leather, microcrystalline wax, synthetic fibers, natural fibers, and mixtures thereof. In one embodiment the non-adhesive substrate is selected from the group consisting of non-adhesive cellulose derivatives, polyester, polypropylene, nylon, rayon, cloth, paper, microcrystalline wax, and mixtures thereof. In another embodiment the non-adhesive substrate is selected from the group consisting of polyester, polypropylene, rayon, nylon, cloth, paper, and mixtures thereof; in another embodiment is polyester.

In one embodiment the non-adhesive self-supporting substrate may be in any physical form suitable for providing strength and/or integrity to the present denture adhesive component and/or water insoluble thermoplastic component. Such physical forms include extruded films and/or sheets, non-woven, woven, continuous, chopped, foam, and combinations thereof. In addition, the non-adhesive self-supporting layer may be formed by any process commonly known in the art. Such processes include extrusion, casting, calendaring, coating, un-bonded, spraybonded, spun-bonded, needle-punched, carded, thermal bonded hydroentangled, meltblown, aperture print bonded, needled, wet-laid, dry-laid, and combinations thereof.

In one embodiment the non-adhesive self supporting substrate has a flexural stiffness of less than about 10 grams/cm, in another embodiment less that about 5 grams/cm, in another embodiment less that about 3 grams/cm, in another embodiment less than about 2 grams/cm and in yet another embodiment from about 0.1, 0.5, 1, to about 2, 3, 5, 10 grams/cm, in any combination, flexural stiffness as measured on a Handle-O-Meter, model #211-300, available from Thwing-Albert Instrument Company of Philadelphia, Pa. as per test method ASTM D2923-95. Flexural stiffness is a material property that is a function of a combination of film/strip thickness, width and material modulus of elasticity. This test is a method for measuring the rigidity of polyolefin film and sheeting. It determines the resistance to flexure of a sample by using a strain gauge affixed to the end of a horizontal beam. The opposite end of the beam presses across a strip of the sample to force a portion of the strip into a vertical groove in a horizontal platform upon which the sample rests. A microammeter wired to the strain gauge is calibrated in terms of deflection force. The rigidity of the sample is read directly from the microammeter and expressed as grams per centimeter of the sample strip width.

### Miscellaneous Additives

### Plasticizing Agent

The articles of the present invention may also optionally comprise a safe and effective amount of one or more toxicologically-acceptable plasticizers. In varying embodiments the level of the plasticizing agent ranges from about 0.01% to about 40%, from about 1% to about 10%, or from about 2% to about 5% by weight of the article. In another embodiment the plasticizer is water insoluble.

Suitable plasticizing agents of the present invention include, but are not limited to, polyols (such as sorbitol); glycerin; propylene glycol; acetylated monoglyceride; hydrogenated starch hydrolysates; corn syrups; xylitol, glycerol monoesters with fatty acids; triacetin; diacetin; monoacetin; dimethyl phthalate; diethyl phthalate; dioctyl phthalate; diethylene glycol; triethylene glycol; tricresyl phosphate; dimethyl sebacate; ethyl glycolate; ethylphthalyl ethyl glycolate; o- and p-toluene ethyl sulfonamide; phthalic acid, glycerol triacetate, citric acid, phosphoric acid, glycol, a pentaerythritol ester of a fatty acid, stearic acid, glycerol monostearate, polyethylene glycol, butyl phthalyl butyl glycolate, dimethyl phthalate, dibutyl phthalate, triacetin, triethyl citrate, acetyl triethyl citrate, acetyl tributyl citrate, triphenyl phosphate, diethylene glycol, caprylic triglyceride, capric triglyceride, propylene glycol dicaprylate/caprate, their derivatives, or combinations thereof.

The articles of the present invention may also optionally comprise a safe and effective amount of one or more toxicologically-acceptable plasticizers. In varying embodiments the level of the plasticizing agent ranges from about 0.01% to about 40%, from about 1% to about 10%, or from about 2% to about 5% by weight of the article. In another embodiment the plasticizer is water insoluble.

Suitable plasticizing agents of the present invention include, but are not limited to, polyols (such as sorbitol); glycerin; propylene glycol; acetylated monoglyceride; hydrogenated starch hydrolysates; corn syrups; xylitol, glycerol monoesters with fatty acids; triacetin; diacetin; monoacetin; dimethyl phthalate; diethyl phthalate; dioctyl phthalate; diethylene glycol; triethylene glycol; tricresyl phosphate; dimethyl sebacate; ethyl glycolate; ethylphthalyl ethyl glycolate; o- and p-toluene ethyl sulfonamide; phthalic acid, glycerol triacetate, citric acid, phosphoric acid, glycol, a pentaerythritol ester of a fatty acid, stearic acid, glycerol monostearate, polyethylene glycol, butyl phthalyl butyl glycolate, dimethyl phthalate, dibutyl phthalate, triacetin, triethyl citrate, acetyl triethyl citrate, acetyl tributyl citrate, triphenyl phosphate, diethylene glycol, caprylic triglyceride, capric triglyceride, propylene glycol dicaprylate/caprate, their derivatives, or combinations thereof.

In another embodiment the plasticizer is water insoluble. In one embodiment the denture adhesive article, when extruded thermoplastically, does not cure and set as a result of the action of the plasticizer component. In another embodiment the plasticizer component does not solidify the viscosity index improver, the optional water insoluble component, and/or the denture adhesive article. Alternatively, in one embodiment the denture adhesive article may be substantially free of plasticizers. In another embodiment, the composition is substantially free of water soluble plasticizers. In one embodiment the oral care composition can be substantially free of polyethylmethacrylate, triacetin, phthalic acid and its derivatives, glycerol triacetate, citric acid and its derivatives, phosphoric acid and its derivatives, glycol and its derivatives, paraffin wax, a pentaerythritol ester of a fatty acid, stearic acid and its derivatives, glycerol monostearate, polyethylene glycol, butyl phthalyl butyl glycolate, dimethyl phthalate, dibutyl phthalate, triacetin, triethyl citrate, acetyl triethyl citrate, acetyl tributyl citrate, triphenyl phosphate, diethylene glycol, caprylic triglyceride, capric triglyceride, propylene glycol dicaprylate/caprate, polyethylene, and glycerin.

### Gellant Agents

The articles of the present invention may also optionally comprise a safe and effective amount of one or more toxicologically-acceptable gellants. In varying embodiments, the level of the gellant agent ranges from about 0.01% to about 40%, from about 1% to about 10%, or from about 2% to about 5%, by weight of the article.

Suitable gellant agents of the present invention include, but are not limited to, polyvinylpyrrolidone/eicosene copolymer sold under the tradename Ganex® V-220F from ISP; tricontanyl polyvinylpyrrolidone sold under the tradename Ganex® WP-660 from ISP; and polyamide gellants including Sylvaclear®, Sylvacote®, Sylvagel®, and Uniclear® all available from Arizona Chemical; or combinations thereof.

### Therapeutic Actives

The denture adhesive articles may also comprise one or more therapeutic actives. Therapeutic actives may be present at a level of from about 1, 5, 10, 15, 20, 25, 30%, to about 3, 5, 10, 15, 20, 30, 50, 70%, or any combination thereof. Therapeutic actives include, for example, antimicrobial agents such as iodine, triclosan, peroxides, sulfonamides, bisbiguanides, or phenolics; antibiotics such as tetracycline, neomycin, kanamycin, metronidazole, cetylpyridinium chloride, domiphen bromide, or clindamycin; anti-inflammatory agents such as aspirin, acetaminophen, naproxen and its salts, ibuprofen, ketorolac, flurbiprofen, indomethacin, eugenol, or hydrocortisone; dentinal desensitizing agents such as potassium nitrate, strontium chloride or sodium fluoride; fluorides such as sodium fluoride, stannous fluoride, MFP (monofluorophosphate); anesthetic agents such as lidocaine or benzocaine; whitening agents such as peroxide; anti-fungals such as those for the treatment of candida albicans; insulin; steroids; herbal and other plant derived remedies; and baking soda. Other suitable therapeutic actives are discussed in the Physicians Desk Reference 62^{nd} Ed., 2008 and the Physicians Desk Reference for non-prescription drugs, dietary supplements, and herbs, 29^{th} Ed. (portions pertaining to non-prescription drugs, dietary supplements, and herbs are hereby incorporated by reference.).

According to one embodiment, the active is selected from the group consisting of: anti-calculus, fluoride ion source, stannous ion source, whitening, antimicrobial, anti-plaque, anti-stain, anti-deposition, anti-gingivitis, anti-tartar, anti-periodontitis, anti-sensitivity, anti-cavity, anti-inflammatory, nutrients, antioxidants, anti-viral, anti-fungal, analgesic, anesthetic, H-2 antagonist, and combinations thereof.

### Other Miscellaneous Additives

Other suitable ingredients include colorants, preservatives (such as methyl and propyl parabens, for example), and rheology modifiers (such as silicon dioxide, for example). Rheology modifiers modify the rheological properties such as viscosity, elasticity, and or yield stress. The colorants, preservatives, and rheology modifiers may be present at levels of from about 0% to about 20%, by weight of the article, in another embodiment from about 0.1%, 0.2, 1, 2, 5, to about 1, 5, 10, 20%, or any combination thereof.

Additionally, the articles may also comprise one or more solvents. These optional solvents may be miscible with the viscosity index improver, water insoluble component, or both, and/or be capable of being dissipated in-situ. In one embodiment these solvents may be dissipated in-situ by evaporation, dissolution, dispersion, bio-absorption, or any other suitable means. In another embodiment, when the denture adhesive composition is an article, these solvents may be dissipated in-situ to leave behind a denture adhesive article. Such solvents may include materials with a viscosity ranging from 0.01, 0.1, 1, 5 centipoise at 20° C., to 5, 10, 100, 1000 centipoise at 20° C. and any combination thereof to make a range, starting point, and/or end point. In one embodiment, solvents include silicones, hydrocarbons, iso-dodecane, iso-hexadecane, iso-eicosane, polyisobutene, or combinations thereof.

### Flavors, Fragrance, Sensates

The articles of the present invention may also include one or more components which provide flavor, fragrance, and/or sensate benefit (warming or cooling agents). Suitable components include menthol, wintergreen oil, peppermint oil, spearmint oil, leaf alcohol, clove bud oil, anethole, methyl salicylate, eucalyptol, cassia, 1-8 menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof, as well as coolants. The coolant can be any of a wide variety of materials. Included among such materials are carboxamides, menthol, ketals, diols, and mixtures thereof. In one embodiment the coolants in the present articles are selected from the group consisting of the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979. These agents may be present at a level of from about 0% to about 40%, in another embodiment from about 0.05 to about 5%, and in another embodiment from about 0.1 to about 2%, by weight of the article.

### Backing Layer and Release Liner

The denture adhesive articles may be provided by itself or may be applied to, coated on, or otherwise provided with a backing layer. The backing layer can be provided as a single layer or as a laminate formed from a plurality of layers, such as any combination of a foam, mesh, and/or other suitable material. The backing layer can be water permeable, water impermeable, partially water permeable, water soluble, water insoluble, erodible, or a combination thereof. Additionally, the backing layer can be continuous or discontinuous (for example, formed from a plurality of discrete segments).

In one embodiment, the backing serves as a protective barrier for the adhesive and/or active. The barrier prevents substantial leaching and/or erosion of the adhesive and/or active by, for example, the wearer's lips, tongue, cheek, as well as saliva. This allows the denture adhesive article to adhere to the oral surface and/or denture for an extended period of time, from several minutes to several hours.

The backing may comprise polymers, natural and synthetic woven materials, non-woven material, foil, paper, rubber, and combinations thereof. The backing may be a single layer of material or a laminate of more than one layer. Preferably, the material is any type of polymer or combination of polymers that have flexural rigidity and are compatible with the denture adhesive articles. Suitable polymers include, but are not limited to, polyethylene, ethylvinylacetate, polyesters, ethylvinyl alcohol and combinations thereof. The backing is generally less than about 1 mm thick, preferably less than about 0.05 mm thick, and more preferably from about 0.001 to about 0.03 mm thick. A polyethylene backing is preferably less than about 0.1 mm thick and more preferably from about 0.005 to about 0.02 mm thick. The shape of the backing is any shape and size that covers the desired oral surface.

The backing is held in place on the oral and/or denture surface by adhesive attachment provided by the denture adhesive article. The general tackiness of the denture adhesive article causes the backing to be adhesively attached to the oral and/or denture surface without substantial slippage from the frictional forces created by the lips, teeth, tongue and other oral surfaces rubbing against the backing while talking, drinking, etc.

The denture adhesive articles may also further comprise a release liner. The release liner may be formed from any material which exhibits less affinity (including zero affinity) for the denture adhesive article than the denture adhesive article exhibits for itself and for the backing. In one embodiment, the release liner comprises a rigid sheet of material such as polyethylene, paper, polyester, or other material which is then coated with a non-stick type material.

### Process for Preparation of the Article

In some embodiments, the articles are formed by processes conventional in the arts, e.g. the film-making industries such as casting, coating, calendaring, extrusion. One example of a method for manufacturing includes: a) adding a viscosity index improver and/or water insoluble component to a vessel, b) heating and mixing the viscosity index improver and/or water insoluble component to at least about 55° C., and c) adding and mixing a denture adhesive component. The temperature of the method will need to be adjusted based on the requirements for the viscosity index improver and/or water insoluble component being used. Thereafter, the melted mixture may be poured into molds or cast to an acceptable thickness on an appropriate substrate. Examples of such substrates include Mylar, continuous moving stainless steel belt (which may eventually enter a dryer section if needed), release paper, and the like. The articles are then cooled. The articles may then be dried if needed, e.g. in a forced-air oven. The temperature of the drying air and length of drying time depend on the nature of the solvent utilized, as is recognized in the art. Generally, the drying temperatures include a temperature between about 25° C. and 140° C., in another embodiment from about 60° and 90° C. for a duration of about 20 minutes to about 60 minutes, in another embodiment from about 30 minutes to about 40 minutes. The articles may then be cut into desired shapes with desired dimensions and then stacked and/or subsequently packaged. In one embodiment, after processing, the article is then die-cut into desired shapes. These shapes may facilitate application of the article to the dentures.

Another conventional film-making process known in the art is extrusion. This method is possible with films wherein the film-forming ingredient comprises a variety of extrudable materials. The mechanical particulars of the extrusion process, e.g. the particular equipment utilized, the extruding force, the shape and temperature of the orifice and/or dies, are considered to be within the skill of the art and can be varied in a known manner to achieve the physical characteristics of the articles described herein.

In one embodiment the thickness of the articles herein is generally between about 0.1 mm to about 2.5 mm, in another embodiment from about 0.4 mm to about 1.5 mm thick, and in another embodiment from about 0.5 mm to about 1 mm thick. The article may be thicker or thinner depending on the degree of cushioning desired by the user or wearer.

In one embodiment the articles herein may optionally be multiphase or have visually distinct phases. In another embodiment the articles herein may optionally have a release liner.

In addition to the inventive compositions and articles detailed above, the present invention also relates to methods for manufacturing such compositions. U.S. Pat. No. 6,025,411 describes processes for making denture adhesive creams or liquids. Previous to the present invention, manufacturers of denture adhesive compositions believed that the compositions should be made at a lower temperature, like around 65° C. or less. Additionally, even if the components being used in the compositions required a higher temperature, often due to a higher melt point, the methods of manufacture for these compounds taught that the compositions should be cooled to a lower temperature, again usually 65° C. or less, before addition of an adhesive component. However, in the present invention, processing the adhesive with the water insoluble component and/or viscosity index improver at a higher temperature can deliver a composition with superior properties and/or performance. The components can be processed at a temperature from about 75° C., 80° C., 85° C., 90° C., 95° C., and 100° C. to about 80° C., 90° C., 100° C., 110° C., and 120° C. and/or any combination thereof to define a range, starting point, and/or end point.

In one embodiment, the present invention is directed to a method of manufacturing a denture adhesive composition, comprising: a) adding an adhesive component to a vessel; b) adding a water insoluble component and/or a viscosity index improver to a vessel; and c) heating the water insoluble component and/or viscosity index improver to a temperature of at least about 70° C. In one embodiment, the adhesive component is added at a temperature of at least about 70° C. In another embodiment, the adhesive component is added prior to or during heating of the water insoluble component and/or viscosity index improver. In an additional embodiment, the adhesive component is added to the vessel below the melting point of the water insoluble component and/or viscosity index improver.

In multiple embodiments, the water insoluble component and/or viscosity index improver is heated to a temperature from about 55° C., 60° C., 65° C., 70° C., 75° C., 80° C., 85° C., 90° C., 95° C., and 100° C. to about 80° C., 90° C., 100° C., 110° C., and 120° C. and/or any combination thereof to define a range, starting point, or ending point. In multiple embodiments, the adhesive component is added at a temperature from about 55° C., 60° C., 65° C., 70° C., 75° C., 80° C., 85° C., 90° C., 95° C., and 100° C. to about 80° C., 90° C., 100° C., 110° C., and 120° C. and/or any combination thereof to define a range, starting point, and/or end point. Additionally, in another embodiment, the adhesive component is added at or below room temperature.

In another embodiment, the water insoluble component and/or viscosity index improver is heated to a temperature of at least about 75° C. and the adhesive component is added when the water insoluble component and/or viscosity index improver reaches its melting point. In an additional embodiment, the water insoluble component and/or viscosity index improver is heated to a temperature of at least about 90° C. and the adhesive component is added when the water insoluble component and/or viscosity index improver reaches its melting point. In another embodiment, at least one adhesive component is combined with at least one water insoluble component and/or viscosity index improver at a temperature at least about 20° C. higher than the melting point of the water insoluble thermoplastic component. In one embodiment, the adhesive component is added to the water insoluble component and/or viscosity index improver at a temperature below the melting point of the water insoluble component and/or viscosity index improver. In another embodiment, the water insoluble component and/or viscosity index improver has a melting point above about 75° C. and the adhesive component is added at a temperature below the melting point of the water insoluble component and/or viscosity index improver.

In one embodiment, a method of manufacturing a denture adhesive composition comprises the steps of mixing at least one of the denture adhesive components with the viscosity index improver at a temperature above about 10° C. below the melting point of the viscosity index improver and subsequently cooling the composition to about 10° C. below the melting point of the viscosity index improver, wherein the composition comprises from about 10.0% to about 60.0% of a denture adhesive component comprising AVE/MA, salts of AVE/MA, mixed salts of AVE/MA, carboxymethylcellulose, or a combination thereof; from about 0.001% to about 30.0% of a viscosity index improver selected from the group consisting of microcrystalline wax, polyethylene, rubber, elastomers, and a combination thereof; and from about 20.0% to about 90.0% of a water insoluble component.

In one embodiment, the composition may comprise from 0.001% to about 30.0% of a viscosity index improver comprising microcrystalline wax, from 10.0% to about 60.0% of a denture adhesive component comprising a mixed salt of AVE/MA and carboxymethylcellulose, and from about 20.0% to about 90.0% of a water insoluble component comprising mineral oil, wherein to manufacture, only the carboxymethylcellulose is mixed with the viscosity index improver at a temperature above about 10° C. below the melting point of the viscosity index improver, and the mixed salt of AVE/MA is added and mixed after cooling the composition to about 10° C. below the melting point of the viscosity index improver. In another embodiment, the same composition may be made wherein the carboxymethylcellulose is mixed with the viscosity index improver at a temperature above about 10° C. below the melting point of the viscosity index improver and the mixed salt of AVE/MA is then mixed with the carboxymethylcellulose and viscosity index improver before the cooling step. In yet another embodiment, the same composition may be made wherein the mixed salt of AVE/MA is mixed with the viscosity index improver at a temperature above about 10° C. below the melting point of the viscosity index improver, cooling the mixed salt of AVE/MA and viscosity index improver to about 10° C. below the melting point of the viscosity index improver, and then adding the carboxymethylcellulose.

In another embodiment, the method of manufacturing further comprises mixing the adhesive component and the water insoluble component and/or viscosity index improver. In one embodiment, at least part of the mixing takes place under vacuum. In one embodiment, the method further comprises cooling the denture adhesive composition. In another embodiment, the method further comprises cooling the water insoluble component and/or viscosity index improver prior to adding the adhesive component. In a further embodiment, the act of cooling can be accomplished by removing the heat source. In another embodiment, the act of cooling is accomplished by actively cooling. Active cooling includes, for example, a water bath, cooling jacket, air cooling (like by blowing air on the composition, for example), etc.

In an additional embodiment, the method further comprises adding an additive to the vessel. In one embodiment the additive is selected from the group consisting of: rheology modifiers, flavorings, colorants, and mixtures thereof. The additive may be added at any time during the manufacturing of the denture adhesive. In another embodiment, the method further comprises dispensing the denture adhesive composition into a container.

In one additional example of a method for manufacturing includes: a) adding a viscosity index improver and/or water insoluble component to a vessel, b) heating and mixing the viscosity index improver and/or water insoluble component to at least about 55° C., and c) adding and mixing a denture adhesive component.

While the exact mechanism is not fully understood, it is believed that the viscosity index improver interacts with the denture adhesive component in such a manner that, in some embodiments comprising a water insoluble thermoplastic component such as petrolatum and a viscosity index improver such as microcrystalline wax blended into mineral oil at temperatures higher than about 75, 80, 85, 90, or 95° C. (and the denture adhesive components are also present in the mix at temperatures above about 75, 80, 85, 90, or 95° C.) can regulate the uptake of saliva and/or exhibit other benefits described above.

### Article Use

The present articles are generally applied to the denture prosthesis and thereafter the denture is secured to the oral cavity. In one embodiment the dentures are dried prior to application of the article. In one embodiment it is not necessary to wet the article and/or the denture prosthesis prior to applying it to the denture prosthesis in order to make the article stick to the denture prosthesis. The article may be applied to any suitable location on the prosthesis. In one embodiment the denture wearer generally wears the article from about 1 hour to about 3 days, in another embodiment from about 6 hours to about 24 hours. After usage the prosthesis is removed from the oral cavity, and any remaining part of the article may be cleaned from the prosthesis, for example by gentle scrubbing with water and a brush. In one embodiment the articles can be combined into a kit, and the kit can further comprise an indicia which indicates to the user not to wet the product prior to application to the denture or oral surface.

### Data

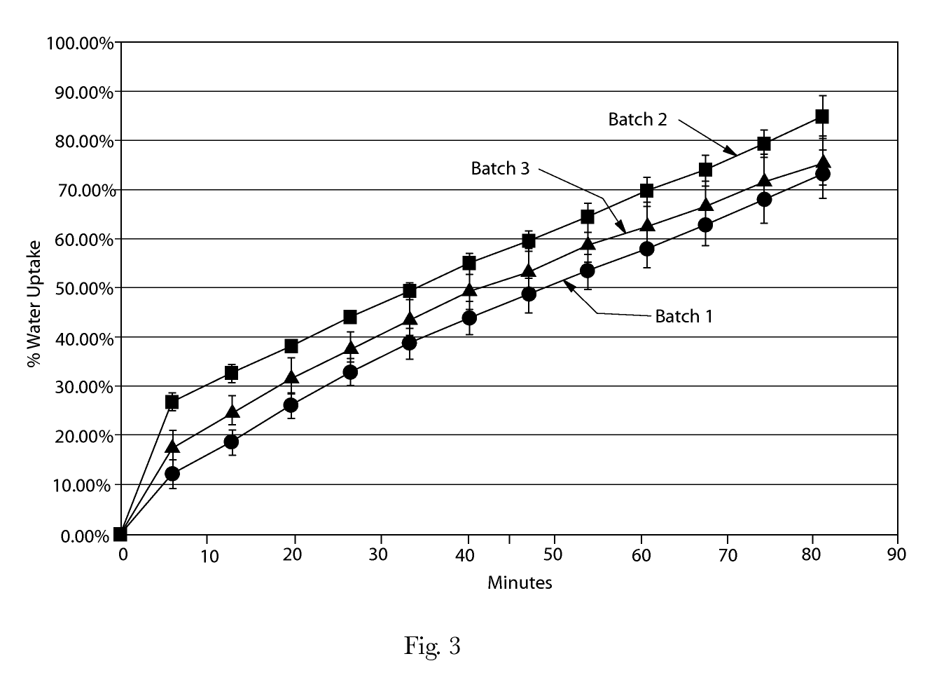

FIG. 3, a chart of % water uptake, demonstrates that viscosity index improvers (in this case microcrystalline wax) are capable of nucleating and coating the denture adhesive, thus reducing % water uptake of the denture adhesive. The chart compares the % water uptake of three batches of denture adhesive with the same formula (6% microcrystalline wax W-835 (MCW), 33% Gantrez (Ca—Zn), 20% carboxymethylcellulose (CMC), 1.14% silica, and 39.86% mineral oil), that are made differently. For batch 1, the MCW was completely melted at 95° C., mixed with mineral oil at 95° C., mixed with Gantrez/CMC/silica at 95° C., and subsequently cooled to 65° C. The second batch was made similarly to batch 1, except the entire process was conducted at 65° C. The third batch was made similarly to batch 1, except only the MCW was melted at 95° C., but subsequently injected into mineral oil at 65° C., after which the GS/CMC/silica was mixed in at 65° C.

Each of the batches was then immersed into a bath of artificial saliva and measured how much artificial saliva was taken up over time [using the “Automated Denture Adhesive Hydration Rate Test” method]. This is indicated as % water uptake in FIG. 3.

The data shows that, for batch 1, the melting of the microcrystalline wax at 95° C., mixing with mineral oil at 95° C., and mixing with Gantrez/CMC/silica also at 95° C., and then subsequently cooling to 65° C. allows the microcrystalline wax to nucleate and coat the Gantrez/CMC, as shown by the comparatively low % water uptake. The % water uptake of batch 1 is the lowest of the three batches, which would be desirable for a denture adhesive, given that lower % water uptake means more resistance to excessive hydration. In comparison, the % water uptake of batch 2, which was made entirely at the cooler temperature, is much higher, indicating that the microcrystalline wax was not capable of nucleating or coating the Gantrez/CMC. The % water uptake of batch 3, in which only the microcrystalline wax was added at the higher temperature, is in between that of batch 1 and batch 2, indicating that the microcrystalline wax only able to do some incomplete nucleating and coating of the Gantrez/CMC.

The following “% adhesive retained” data demonstrates that viscosity index improvers (in this case microcrystalline wax) are capable of improving the % adhesive retained. The data compares the % adhesive retained of two denture adhesive formulas, one with a viscosity index improver and one without. Formula A, with a viscosity index improver, contained 33% Ca/Zn Gantrez salt, 20% CMC, 1.14% silica, 39.86% mineral oil, and 6.0% microcrystalline wax (W-835). Formula B, without a viscosity index improver, contained 33% Ca/Zn Gantrez salt, 20% CMC, 1.14% silica, about 23.93% mineral oil, about 21.87% petrolatum, and 0.06 colorant. Both formulas were characterized using the “Adhesive Retention Method” described below.

Formula A (with a viscosity index improver) retained 82% of the adhesive, while Formula B (without a viscosity index improver) retained only 52% of the adhesive. This demonstrates that using a viscosity index improver (microcrystalline wax in this case) improves the % adhesive retained.

### Test Methods

To measure the instant viscosity ratio for a given material, for example RS or PS, one calculates the ratio of the material's viscosity at room temperature (25° C.) to its viscosity at an elevated temperature (40° C.), using the following procedure:

### Equipment:

Ares Strain-Controlled Rheometer
25 mm permanent parallel plates

### Method:

1. Load 25 mm parallel plates onto an Ares rheometer.
2. Zero the normal force.
3. Zero the gap @ 25° C. (i.e. room temperature).
4. Apply the sample of the material to the bottom plate in a semi circular motion moving across the plate. There should be enough specimen such that when a gap of 2.177±0.005 mm is reached and excess is trimmed, the specimen extends evenly to all edges of the plate with no gaps present.
5. Adjust the Gap using the following procedure:
   Click on set gap icon. Set command gap position to 2.55 mm.
   Set the Max Force Allowed to 100 g.
   Click on set Gap.
   Trim sample with plastic cover slide.
   Set the command gap position to 2.177 mm, Max Force Allowed=100 g.
   Click on set Gap.
   Trim sample with plastic cover slide.
   Set command gap position to 2.147 mm. Max Force Allowed=100 g.
   Click on set Gap.
   Do Not Trim Sample.
   Final Gap should read 2.147±0.005 mm
   Allow the temperature to equilibrate to 25° C.
   Record the Gap and the Axial Force in test notes along with any observations made.
   Start Experiment
6. Start test:
   Method is a Step Rate (Transient) test that runs the following procedure:
      i. Applies a rate of 0/s for 1 s (a 1 s delay)
      ii. Applies a rate of 5/s for 5 s
   Result should be a curve of Viscosity vs. Time
7. Record the peak viscosity (aka “Instant Viscosity”) of this curve.
8. Repeat steps 1-7 for the material at 25° C.—a minimum of three times
9. Repeat steps 1-7 for the material at 40° C.—a minimum of three times
10. Calculate the average value of the Instant Viscosity for the material at 25° C., and separately at 40° C.
11. Finally, calculate

“Instant Viscosity Ratio”=(Average Instant Viscosity for the material at 40° C.)/(Average Instant Viscosity for the material at 25° C.).

### Saliva Penetration Ratio Test Method

The saliva penetration ratio can be measured by the following procedure:

### Materials:

Cylindrical probes, 1″ tall×1″ diameter, made from polymethylmethacrylate
Artificial saliva (described in earlier section)
50-ml glass beakers
Oven
Balance

### Method:

1. The Prototype Sample and Reference Sample are prepared using the procedure described earlier.
2. Each probe is pre-weighed to get a clean weight.
3. About 0.50 g of the sample is applied to the surface of the probe.
4. The probe is submerged in a 50-ml beaker containing 30-ml of artificial saliva at 40° C. (close to mouth temperature).
5. The beaker is placed in a 40° C. oven.
6. The beaker is removed from the oven after 20 minutes. The probe is removed from the beaker and gently shaken to remove loose/excess artificial saliva and weighed.
7. Calculate

“Saliva Penetration” of sample=(Weight of Probe+Saliva) after 20 minutes−(Initial weight of probe only)
8. Repeat steps 1-7 for the Reference Sample and Prototype Sample—a minimum of three times each.
9. Calculate the average value of the “Saliva Penetration” for the Prototype Sample, and separately for the Reference Sample.
10. Finally, calculate

“Saliva Penetration Ratio”=(Average Saliva Penetration of Prototype Sample)/(Average Saliva Penetration of Reference Sample)

### Vapor Penetration Ratio Test

The Vapor Penetration Ratio can be measured by the following procedure:
1. Set chamber at 25° C.—for example the Surface Measurement Systems DVS-2000 Dynamic Vapor Sorption Analyzer.
2. Prepare Sample as follows: Squeeze out about 1.0 g of sample and discard. Carefully dispense 100-110 mg of denture adhesive into an inverted plastic GC (gas chromatography) vial cap and smooth sample into an even layer with a small spatula or similar tool.
3. Place empty GC vial cap in the reference side of the DVS 2000 (to zero out contribution from cap). Place prepared sample in the sample chamber of the DVS 2000.
4. Set the humidity to zero initially, followed by a step change to 90% relative humidity after 15 minutes.
5. Record weight gain automatically over 12 or more hours.
6. Repeat steps 1-5 for the Reference Sample and Prototype Sample—a minimum of three times each.
7. Calculate the average value of the “Weight Gain” for the Prototype Sample, and separately for the Reference Sample.
8. Finally, calculate

“Vapor Penetration Ratio”=(Average Weight Gain for the Prototype Sample)/(Average for the Reference Sample) 1.

### Ooze Ratio Test

The Ooze Ratio can be measured and calculated by the following procedure:

### Equipment:

Instron 5544 enclosed in a Plexiglas case equipped with a heating element to control internal temperature.
Cylindrical probes, 1″ diameter, made from polymethylmethacrylate

### Procedure:

1) Set the temperature to about 40° C.
2) Load the initial sample weight of about 0.50 grams uniformly onto a 1″ cylindrical probe.
3) Bring the probe to within 1.2 mm of the base plate, also made from polymethylmethacrylate.
4) Allow the sample to equilibrate for about 10 minutes.
5) Apply 200 gram-force for 90 seconds.
6) At 90 seconds, trim and weigh material that has oozed out (aka “Ooze”).
7) Repeat steps 1-6 for the Reference Sample and Prototype Sample—a minimum of three times each.
8) Calculate the average value of “Ooze” for the Prototype Sample, and separately for the Reference Sample.
9) Finally, calculate

“Ooze Ratio”=(Average Ooze for the Prototype Sample)/(Average Ooze for the Reference Sample)

### Light Scattering Ratio Test

The Light Scattering Ratio can be measured and calculated according to the following procedure which uses a phase technology method:

A phase technology method distinguishes between the temperature-resistance of different crystalline and/or opaque structures in a water-insoluble component phase of various denture adhesive compositions.

### Equipment:

Phase Technology Phase Transition Analyzer Model No. NK60-KPA—This instrument is essentially a light scattering device. The optics source sits above the sample, which sits in a reflective “bowl”. The instrument measures the intensity of the light that is scattered by the sample. It is similar in principle to a light transmittance device except that it measures the intensity by reflectance instead of transmittance. This instrument can also be used to measure the Cloud Point temperature.

### Method:

1. 0.16 g specimens are weighed into the Phase Technology instrument.
2. The specimen is first subject to a temperature ramp as follows
   a. About 20° C. to about 65° C. and back to about 20° C. at about 5° C. per minute
   b. This first ramp is used solely to remove air bubbles and smooth out the sample surface. The data generated is not used.
3. The sample is then subject to a second, identical temperature ramp:
   a. About 20° C. to about 65° C. and back to about 20° C. at about 5° C. per minute
   b. During this ramp a plot of light scattering or “opacity” vs. temperature data are generated and this data set is recorded and saved.
4. Load the data from this second heating curve into kaleidagraph (Synergy Software Version 3.5) and integrate the area under this curve from 20° C. to about 65° C.
5. Repeat steps 1-4 three times, for the prototype wic (water insoluble component) and reference wic. Calculate the average. Note: The procedure to prepare the prototype and reference samples are described in previous sections. To prepare the wic's, follow the procedure to prepare the samples, but omit the steps to add the denture adhesive components and excipient Powders.
6. Calculate “Light Scattering Ratio”=(Average Area Under Curve of Prototype wic)/(Average Area Under Curve of Reference wic).

### Penetration Value Test

Unless otherwise noted, the term “Penetration Value” as used herein refers to the “Needle Penetration Value” which is measured using ASTM D-1321; if the method ASTM D-1321 is found to be substantially not suitable to measure the Penetration Value of certain materials, the “Cone Penetration Value” as measured by ASTM D-937 can be used instead.

The bioerosion of the inventive articles can be measured by the following method: run a water source on top of the sample specimen for about 30 minutes while the specimen sits atop a wire mesh. The water source is a laboratory faucet adjusted such that the temperature is 39±1° C. and the flow rate is 16±1 ml/sec. Use a funnel to focus the flow and help dampen the effect of small pressure and temperature fluctuations within the water lines. The wire mesh grid has square openings approximately 0.09 inches×0.09 inches and is placed 2.5 inches below the tip of the funnel where it is clamped to a metal ring for support. Sample specimens weighing 0.025 g are placed on the mesh and images are taken at 0, 10 and 30 minutes to follow bio-erosion of the specimen. After 30 minutes the wire mesh containing the remainder of the specimen is removed and heated for 1 hour at 60° C. under vacuum to remove all remaining water. After the heating period, final weights are taken to calculate weight loss due to bio-erosion. An average of 3 specimens per sample are used to calculate bio-erosion time and weight loss. The article is bioerodible if it does not leave behind visible residue, film, or sheet after about 30 minutes under these testing conditions, and/or if it cannot be easily separated or peeled away manually in one or more large pieces after about 30 minutes under these testing conditions, and/or if it leaves behind less than about 2, less than about 4, less than about 6, and/or less than about 8% by weight of residue (of the original weight of the article) after about 30 minutes under these testing conditions. The above bio-erosion test may also be conducted at various time-points up to 8 hours.

The dry tack can be measured by the following method: 1. remove the article from the package material; 2. place the article on the palate-portion of a dry, acrylic upper-denture with the teeth facing downward; 3. apply pressure with fingers for about 3 to 10 seconds; 4. thereafter remove finger pressure; 5. then invert the denture with the teeth facing upward. In one embodiment the article demonstrates dry tack if: i. The article does not stick to fingers during steps 1-2, ii. leaves little or no residue on the fingers in steps 3-4, and iii. in step-5, the article does not fall off of the denture, once inverted, for at least about 10-30 seconds, or at least about 1 minute.

In another embodiment the article demonstrates dry tack if: i. The article does not stick to fingers during steps 1-4, and ii. in step-5, the article does not fall off of the denture, once inverted, for at least about 10-30 seconds, or at least about 1 minute.

In another embodiment the article demonstrates dry tack if in step-5, the article does not fall off of the denture, once inverted, for at least about 10-30 seconds, or at least about 1 minute.

The dry tack of the inventive articles can also be measured by the following procedure:

(a) Compress a 5 mm diameter disc (0.67 mm thick) sample of the article between a 1″ diameter cylindrical probe (made from polymethylmethacrylate) and a flat sheet of polymethylmethacrylate with a 2000 gram-force for 2 seconds; (b) Pull off the probe at 1 mm/second and record peak force; (c) Repeat procedure with no sample sandwiched between the two surfaces; and (d) Calculate: Dry Tack in grams/square centimeter=(Peak Force with Sample−Peak Force without sample)/Cross sectional area of sample disc.

In one embodiment, the above procedure is repeated with an applied force of 250 gram-force in step (a) and the tack measured in steps (b)-(d);

The article has dry tack if the tack measured with a 250 gram-force applied force is less than about 25, 50, 100, 200, or 500 grams/square-centimeter, and the tack measured with a 2000 gram-force applied force is greater than about 200, 500, 1000, 2000, 5000, 10000, or 25000 grams/square-centimeter, and any combination of these levels.

The loss modulus G″ and storage modulus G′ of the inventive articles can be measured by the following procedure:

(a) Load a sample disc of 8 mm diameter and 0.67 mm thickness onto a ARES rheometer using a parallel plate fixture with a compressive force of 500 grams; (b) Set strain to be 0.02%; (c) Measure G″ and G′ at a sweep of frequencies including 1 Hz.

The normalized dislodgement force and dislodgement force ratio of the inventive article can be measured by the following method:

Instrument: An Instron model 5544 is used. The load cell is calibrated according to manufacturer's specifications annually. The choice of load cell is determined by having the forces generated by the adhesive fall within the recommended operating range for the load cell. This is typically between 10%-90% of full capacity.

Test Fixtures The geometry of a cylindrical probe and a flat plate are used as the test fixtures. The probe is made from PMMA, 0.2 sq·cm to 10 sq·cm in surface area. For the base plate, the same PMMA material is used but in sheet form, ¼″ thick. This is cut into 6″×6″ plates to be clamped onto the Instron.

Hydrating Liquid: Artificial saliva containing low levels of various salts is used to hydrate the adhesive.

### Artificial Saliva Composition

| Ingredient | Amount per Liter | |
|---|---|---|
| K₂HPO₄ | 4.2 | g |
| KH₂PO₄ | 3.2 | g |
| KOH | 2 | pellets |
| Mineral Stock Solution | 5 | ml |

| | | |
|---|---|---|
| KCl Solution | 8 | g per 100 ml of Stock |
| NaCl | 8 | g |
| Na2SO4 | 0.264 | g |
| MgCl2•6H2O | 0.7687 | |
| | (or 0.36 | g Anhydrous MgCl2) |

Adhesive: 0.1 to 1.0 gram of adhesive is applied to the probe.

Hydration: The hydrating liquid (0.2 mL of artificial saliva to 2.0 ml) is pipetted onto the surface of the adhesive. The assembly is then permitted to hydrate for 20 minutes or more.

Test Method: Once the sample is hydrated, it is mounted onto the Instron and the test is carried out via computer control. The method is comprised of the following steps:

(a) Compression to 750 to 7500 g of force; (b) Hold at compression for 2 minutes; (c) Reduce compressive force to 200 g_{f}; (d) Hold (1 minute); (e) Pull off at 1 mm/s; (f) Record Peak Dislodgement Force; (g) Calculate “Normalized Dislodgement Force”=(Peak Dislodgement Force)/(Surface Area of Probe); report in grams force per sq·cm; (h) Repeat steps (a)-(g) for commercial Fixodent Original denture adhesive (available commercially manufactured by P&G), or for the following reference formula: Ca(47.5%)/Zn(17.5%) MVE/MA salt 33%, sodium carboxymethylcellulose 20%, mineral oil USP (65-75 cst at 40 C) 23.93%, petrolatum USP (consistency 17-20 mm) 21.87%, colloidal silicon dioxide 1.14%, and Opatint OD1646 0.06%; suitable methods to make this reference formula are disclosed in U.S. Pat. No. 5,073,604, Holeva K., and U.S. Pat. No. 6,617,374 Rajaiah J.; (i) Calculate “Dislodgement Force Ratio”=(Peak Dislodgement Force of Prototype Adhesive)/(Peak Dislodgement Force of Fixodent Original or the reference formula described above.

Data: Each sample is repeated a minimum of 3 times and the average value of the “Normalized Dislodgement Force” and “Dislodgement Force Ratio” are reported.

Specifically the normalized dislodgement force and dislodgement force ratio can be measured by using the following parameters in the procedure: 0.25 gram adhesive; 1 inch diameter probe; hydration time of 20 minutes; and compression force of 7500 grams.

The “normalized ooze amount” and “ooze ratio” of the inventive article can be measured by the following procedure:

(a) Load initial sample weight of about 0.50 grams uniformly onto a 1 inch diameter cylindrical probe made from polymethylmethacrylate; (b) Bring probe to 1.2 mm of base plate, also made from polymethylmethacrylate; (c) Apply 750 gram force for 90 seconds; (d) At 90 seconds, trim and weigh material that has oozed out; (e) Calculate “Normalized Ooze Amount”=(Amount oozed out/Initial sample weight)×100; (f) Repeat Steps (a)-(e) using commercial Fixodent Original a denture adhesive cream commercially manufactured by P&G, or with the following reference formula: Ca(47.5%)/Zn(17.5%) MVE/MA salt 33%, sodium carboxymethylcellulose 20%, mineral oil USP (65-75 cst at 40 C) 23.93%, petrolatum USP (consistency 17-20 mm) 21.87%, colloidal silicon dioxide 1.14%, and Opatint OD1646 0.06%; (g) Calculate “Ooze Ratio”=Normalized Ooze Amount of Prototype Adhesive/Normalized Ooze Amount of Fixodent Original or the reference formula described above; (h) Each sample is repeated a minimum of 3 times and the average value of the “Normalized Ooze Amount” and “Ooze Ratio” are reported.

### Automated Denture Adhesive Hydration Rate Test

This describes the procedure for an automated hydration test designed to monitor the uptake of water into samples of denture adhesive. The resulting data can be used to analyze the effect of formulation and processing changes on the ability of denture adhesive to absorb water.

### Materials & Equipment

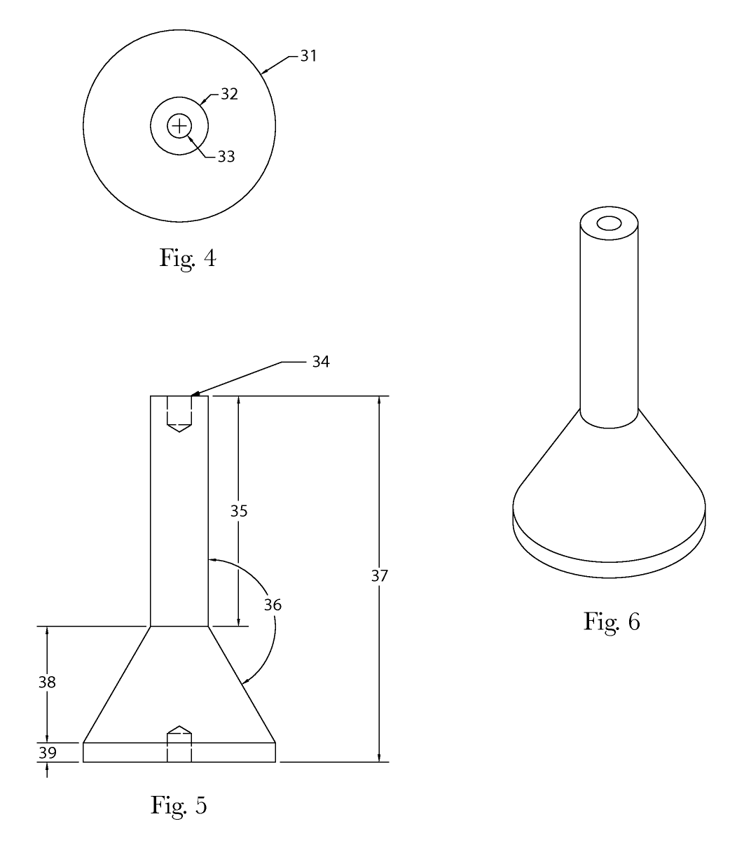

TA.XT Texture Analyzer with 5 kg Load Cell (Manufactured by Stable Micro Systems, inc., UK)
800 ml Beaker (cylindrical, ˜131 mm deep and 94 mm Inner Diameter)
260 g per run of Artificial Saliva(AS) (without mucin)
IKA RCT basic hotplate with magnetic stirrer and ETS-D5 temperature controller and probe
Polytetrafluoroethylene(PTFE) Sample Spindle Assembly, as shown in FIGS. 4-12.
Light Mineral Oil
Laboratory Stand
Chain Clamp (for 800 ml beaker)
Block clamp or 3 prong clamp for ETS-D5 control probe
Denture Adhesive Cream sample
Magnetic Stir Bar
   Note: It should be possible to replicate results without the specific pieces of equipment employed. Any strain controlled mechanical tester with similar capabilities to a TA.XT texture analyzer could be substituted. The same is true of the IKA basic hot plate. Any hotplate with a feedback based temperature control probe and tunable magnetic stirrer RPM could be substituted for the one listed above. This test is geometry sensitive, so altering the spindle geometry will alter numerical results, though results should be similar. It would also be possible to use this test to measure the uptake of test fluids other than artificial saliva.

### Setup

In this experiment the texture analyzer controls each run and records results, while the hot plate provides constant temperate and mild mixing of an artificial saliva bath. The hot plate is positioned directly on the texture analyzer test platform so that the spindle axis is aligned with the center of the hot plate. The 800 ml beaker is positioned in the center of the hotplate. The laboratory stand and clamps are used to position and secure the beaker and temperature probe. The temperature probe hangs into the 800 ml beaker, but should be position such that it will not interfere with the movement of the spindle or contact the adhesive as it swells. The spindle assembly is attached to the texture analyzer such that a ring shaped sample of adhesive loaded into the broad end of the spindle assembly is raised out of and plunged into the warm AS bath as the texture analyzer (or similar instrument) changes the elevation of the loadcell.

### Procedure

1. Ensure that the TA.XT Texture Analyzer load cell is properly calibrated. Recalibrate if necessary.
2. Position hot plate, empty beaker, and temperature probe. Beaker should be centered with both hot plate and spindle.
3. Calibrate Probe Height with empty Spindle and empty beaker
   a. This calibration ensures that the texture analyzer height is zero when the tip of the spindle assembly is just in contact with the bottom of the beaker. When the texture analyzer is in tension mode, positive height values will indicate the distance between the spindle tip and the bottom of the beaker.
4. Set data acquisition time to 5 points per second.
5. Begin a test
   An outline of the test program is provided below. The user is required to carry out the tasks described when prompted. All other tasks should be programmed into the test procedure of the mechanical tester used.
1. Move to a starting position of +125 mm at 10 mm\ Sec
2. Prompt User—“Remove the empty spindle from the Texture Analyzer, then click ok.”
3. Zero load cell
   a. While the load cell is zeroing the user should fill the beaker with 260 g of AS solution.
4. Prompt User—“AS bath should be in position at 40° C. Put in stir bar at 50 rpm. Click Ok when AS bath is ready.”
5. Prompt User—“Wipe down spindle with mineral oil. Wipe off Excess with fresh paper towel. Click Ok.”
   a. A light coating of mineral oil is used in combination with the Teflon spindle to prevent wetting of the sample fixture with water, thus eliminating a potential source of error.
6. Prompt User—“Attach the empty spindle, to TA.XT.Plus then click ok.”
7. Wait for time of 20 Sec.
   a. Data capture on to weigh empty spindle
8. Zero out load cell with spindle
9. Wait for time of 20 Sec.
   a. Data capture on to Collect baseline weight, it should be within instrument error of zero.
10. Prompt User—“Remove the spindle and load with adhesive. Attach the Spindle, then click ok.”
   a. Adhesive is loaded by squeezing adhesive directly onto the spindle from a tube. The sample area is the shelf between the edge of the larger diameter spindle and Teflon spacer. A slight excess of adhesive is used to fill the space created between the main spindle shaft and cap by the spacer. The cap is then screwed down until it is finger tight against the spacer. Excess adhesive is removed with a non-scratching spatula such that the surface of the adhesive is flush with the outside of the sample. A paper towel is used to remove excess adhesive on the outer surfaces that cannot be removed easily with a spatula. The final sample is a flat ring of adhesive of which only the outside edge parallel to the axis of the spindle is exposed.
11. Wait for time of 20 Sec.
   a. Data capture on to Weigh dry adhesive
12. Move down by 80 mm at 20 mm\Sec.
   a. Data capture on, instrument lowers spindle to 5 mm above AS
13. Move down by 25 mm at 5 mm\Sec.
   a. Data capture on, instrument immerses sample in AS
14. Move down by 8 mm at 5 mm\Sec.
   a. Data capture on, go to final position, 12 mm from bottom, leaves ˜3 mm clearance for stir bar
15. Wait for time of 300 Sec.
   a. Data capture on, monitors buoyancy change of spindle in AS bath
16. Move up by 8 mm at 5 mm\Sec.
   a. Data capture on, begin retracting spindle
17. Move up by 30 mm at 5 mm\Sec.
   a. Data capture on, retract to ˜10 mm above AS
18. Wait for time of 96 Sec.
   a. Data capture on, measures adhesive weight out of solution bath
19. Start a repeat loop of the immersion measurement cycle with data capture on
   a. Move down by 30 mm at 5 mm\Sec.
   b. Move down by 8 mm at 5 mm\Sec.
   c. Wait for Time of 300 Sec.
   d. Move Up by 8 mm at 5 mm\Sec.
   e. Move Up by 30 mm at 5 mm\Sec.
   f. Wait for time of 96 Sec. (measures adhesive weight out of solution bath)
20. End repeating loop after 11 cycles (Data capture on for all 11 loops)
21. Prompt User with end of run notification
   Additional Notes: The above set of test conditions was found to work well for measuring the relative water uptake of various formulations. Changing the test conditions and geometry would provide similar though numerically different results.

### PTFE Sample Spindle Assembly Description and Diagrams

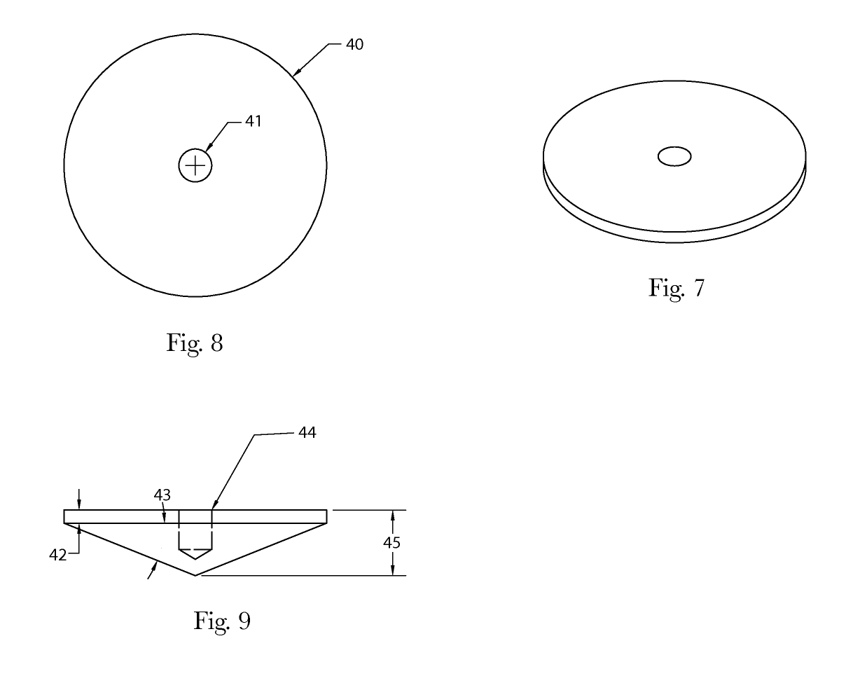

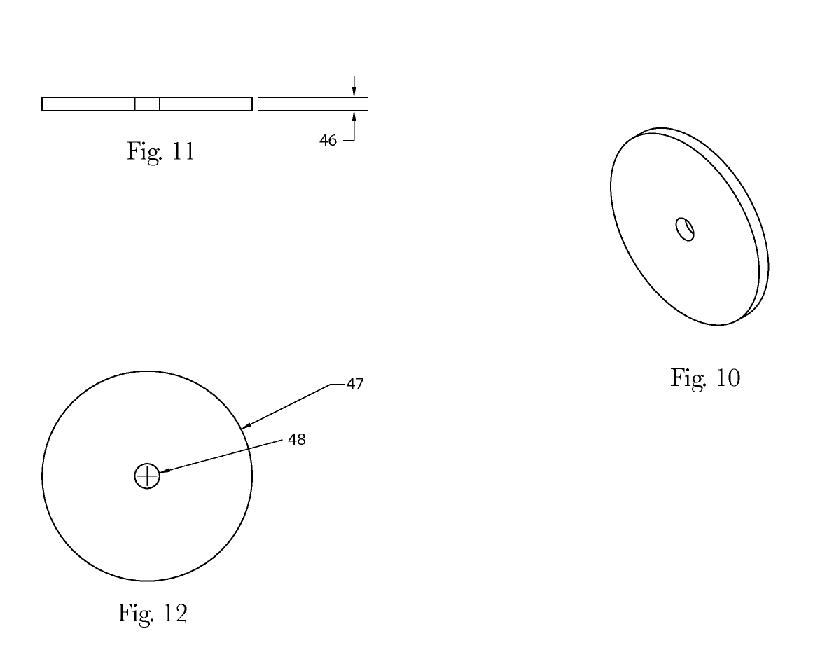

The polytetrafluoroethylene spindle used in this study is composed of three pieces, a main spindle shaft as shown in FIGS. 4-6, a spacer as shown in FIGS. 7-9, and an endcap as shown in FIGS. 10-12.

As shown in FIGS. 4-6, the shaft consists of a rod that broadens into a cone, then a short cylindrical section. This geometry was chosen to minimize weight while also preventing water beads from becoming stuck on the Teflon surface as the spindle is withdrawn from the AS bath during tests. Reference numbers 31-33 reflect the diameters of various positions on the shaft, with diameter 31 being 2.5 inches, diameter 32 being 0.75 inch, and diameter 33 being 0.313 inch. Reference number 34 shows the placement of an M6 insert that presses in and the top and the bottom. Reference number 35 indicates that the height is 3.000 inches, reference 36 shows that the angle is 150°, reference 37 represents the height of 4.766 inches, reference 38 indicates the height is 1.516 inches, and reference 39 indicates that the height is 0.25 inch.

As shown in FIGS. 7-9, the end cap is also a truncated cone, again to minimize the adhesion of water droplets to the spindle assembly. Reference number 40 indicates that the diameter is 2.5 inches, and reference number 41 indicates that the diameter is 0.313 inch. Reference 42 shows a height of 0.125 inch, reference 43 indicates an angle of 22°, reference 44 shows where an M6 insert is pressed in, and reference 45 indicates a height of 0.625 inch.

FIGS. 10-12 show the spindle spacer. Reference number 46 indicates a height of 0.125 inch, reference 47 indicates a diameter of 2.000 inches, and reference 48 indicates a diameter of 0.236 inch.

The assembly is held together and attached to the instrument by screws and thread holes mounted in the cap and shaft. Variations in spindle assembly geometry should not greatly influence results, as long as the volume for the sample created between the shaft and the cap by the spacer is a ring 0.125 inches thick with an inner diameter of 2.0 inches and an outer diameter of 2.5 inches.

### Data Analysis

Data is extracted from the time vs. force plots generated by the texture analyzer. In this experiment the force measured by the load cell is equivalent to sample weight when the sample is not immersed and at rest. Only data from the steps of the procedure when the sample is at rest and out of solution is used in standard tests. It is possible to extract additional information from the buoyancy data recorded while a sample is immersed. Data is reported as the average force value over each period during which the sample is out of solution and at rest. The first and last second of each of these periods is excluded from the data to eliminate inertial effects from the measurements. These forces are taken to be the average sample weight at the average time of each period from the initial immersion of the sample. Final results are reported as water uptake vs. time where water uptake percentage is calculated as:

$$( \frac{current sample wt .}{dry sample wt .} - 1 ) \cdot 100 \%$$

### Adhesive Retention Method

This section describes the procedure designed to evaluate the retention of denture adhesive under shear in a wet environment.

### Equipment & Materials

IKA-WERKE Eurostar power control-visc mixer (any overhead mixer with adjustable RPM is sufficient)
IKA basic hotplate & IKA ETS-D5 temperature probe (any hotplate with a feedback controlled temperature probe is sufficient)
Spray paint, (Black Valspar premium enamel, satin, interior/exterior, fast drying, 65049 or similar)
Custom machined spindle, as shown in FIGS. 4-12.
   ⅜″ steel rod with 1.5″ of threading on one end
   2 Securing nuts (one for each side of the disk assembly)
   2.5″ diameter, 0.125″ thick aluminum top disc with a ¼″ diameter center hole (coated on one side with spray paint)
   2.0″ diameter, 0.125″ thick aluminum spacer disc with a ¼″ diameter center hole (coated on one side with acrylic spray paint)
   2.5″ diameter, 0.125″ thick poly carbonate bottom disc with a ¼″ diameter center hole
Artificial Saliva, 260 g per run

### Sample Preparation

1. Preset to the desired temperature on the ETS-D5 temperature probe and pre-heat the appropriate amount of artificial saliva solution (260 g).
2. Weigh the entire spindle assembly prior to adding adhesive.
3. Slide the aluminum top disc and spacer disc onto the threaded end of the steel rod such that the painted surfaces will face downward towards the bottom of the AS bath when the spindle is loaded into the mixer chuck.
4. Adhesive is spread onto the area 2.5″ diameter aluminum top disc that is not covered by the aluminum spacer disc. Spread the adhesive as evenly as possible to minimize air pockets. The adhesive should only be in contact with the spray painted sides of the top and spacer discs. Excess adhesive should squeeze out of the assembly when the poly carbonate disc is used to sandwich the adhesive.
5. Secure the disc assembly with the securing nuts (one on each side of the tree disc stack).
6. Use a spatula and paper towels to remove excess adhesive from the rim of the disc assembly. When assembled the sample should be sandwiched into a ring 0.125″ thick, with an inner diameter of 2.0″ and an outer diameter of 2.5″. Only the outer edge of the adhesive ring parallel to the spindle shaft is exposed to artificial saliva solution.

### Procedure

1. Check that the artificial saliva bath is preheated to 40° C. and that the beaker is well secured to avoid spills. The temperature probe should be positioned such that the rotating spindle will not contact the probe during a test.
2. Weight the entire spindle and sample. The initial dry adhesive wt.=Adhesive filled spindle wt.−Empty spindle wt.
3. Load the spindle into the mixer chuck and immerse the disc assembly into the artificial saliva solution. The bottom of the poly carbonate disc should align with the beakers 100 ml mark. The axis of rotation of the spindle should align with the central axis of the beaker.
4. Allow sample to hydrate for 10 minutes under mild shear conditions (50 rpm).
5. Remove the sample from the bath and mixer chuck, wick away any excess water with a paper towel, and weight the spindle.
6. Return the spindle to the chuck and AS bath. Set the mixer to a high shear condition (450 rpm) for 40° C., for 10 minutes.
7. Repeat Steps 5 and 6 once more.
8. Remove the sample from the mixer, remove excess water, and weight the sample once more.
9. Disassemble the spindle and carefully scrape all the remaining adhesive into a pre-weighed vial.
10. Determine the weight of the wet adhesive in the vial (Final wet weight).
11. Dry the adhesive overnight (17 hrs.) in a convection oven at 105° C.
12. Weight the dry sample and record the final dried sample weight.
13. Calculate retention values
   a. % of Adhesive Retained=Final dried adhesive wt./Initial Dry adhesive wt.*100%
   b. % Water at end of run=100%−(Final dried adhesive wt./Final wet wt.*100%)
14. Repeat steps 1-13 at least four times and calculate the average % adhesive retained.
   Note: The above procedure is for testing at 40° C. For room temperature studies skip sample preparation step one and record the water temperature. It is also necessary to increase the rpm during the high shear erosion step to between 900 and 1000 rpm. Increasing the rpm's to this value requires increasing the volume of artificial saliva used to ˜800 ml to prevent the liquid vortex from encountering the adhesive. It would be possible to modify this test to examine the erosion behavior of denture adhesive or other similar products that experience shear in an aqueous environment at a variety of temperatures.

### Preparation of Artificial Saliva

The following is a method for preparation of artificial saliva. This method should be used in the Automated Denture Adhesive Hydration Rate Test and in the Disc Rotation Erosion of Adhesive Method.

### Equipment:

1. Mixer
2. Balance

### Materials

1. De-ionized water
2. Potassium Phosphate, Dibasic
3. Potassium Phosphate, Monobasic, anhydrous
4. Potassium Hydroxide
5. Sodium Chloride
6. Sodium Sulfate
7. Potassium Chloride
8. Magnesium Chloride, Hexahydrate

### Procedure

The following components are combined in the order and amounts listed. The resulting mixture was stirred using a high speed mixer for 45 minutes (500-600 rpm) until all the components are dissolved. The finished solution was dispensed into 3 L plastic bottles (e.g. Low density Poly ethylene) and stored at room temperature.

| | | |
|---|---|---|
| | | Amount |
| | Amount per | Per 3 L |
| Components | 1 L Di-H₂O | Di-H₂O |

| | | | | |
|---|---|---|---|---|
| 1. Potassium Phosphate, Dibasic | 4.2 | g | 12.6 | g |
| 2. Potassium Phosphate, Monobasic, | 3.2 | g | 9.6 | g |
| anhydrous | | | | |
| 3. Potassium Hydroxide | 2 | pellets | 6 | pellets |
| 4. Mineral Stock Solution | 5 | ml | 15 | ml |

| | Amount per | |
|---|---|---|
| Component of Mineral Stock Solution | 100 ml | |
| a. Potassium Chloride | 8 | g |
| b. Sodium Chloride | 8 | g |
| c. Sodium Sulfate | 0.264 | g |
| d. Magnesium Chloride, Hexahydrate | 0.7687 | g |

### EXAMPLES

The following examples further describe and demonstrate embodiments within the scope of the present invention. The examples are given solely for the purpose of illustration and are not to be construed as limitations of the present invention. Many variations of these are possible without departing from the spirit and scope of the invention.

### Example I

| | A | B | C | D | E | F | G | H |
|---|---|---|---|---|---|---|---|---|
| | % | % | % | % | % | % | % | % |
| Ca/Zn | 33.00 | 33.00 | 33.00 | 53.00 | | 33.00 | 28 | 24.5 |
| AVE/MA | | | | | | | | |
| Salt | | | | | | | | |
| CMC | 20.00 | 20.00 | 20.00 | | 53.00 | 25.00 | 15 | 28.5 |
| AVE/MA Acid | | | 1.00 | | | | | |
| S-97 | | | | | | | | |
| Micro- | 46.92 | 47.00 | 45.24 | 47.00 | 47.00 | 42.00 | 45.52 | 46.92 |
| crystalline | | | | | | | | |
| Wax¹ W-835 | | | | | | | | |
| Flavors | | | 0.50 | | | | 0.4 | |
| Sacharrin | 00.08 | | 0.16 | | | | 0.08 | 0.08 |
| Colorants | | | 0.10 | | | | | |
| Silica | | | | | | | 1 | |
| Corn Starch | | | | | | | 10 | |

¹Multiwax W 835 manufactured by Witco (Crompton, Sonneborn). The Multiwax W 835 may also be substituted with either Polyethylene A-C 1702 or Polyethylene A-C 6702.

The Microcrystalline Wax W-835 (or Polyethylene AC 1702 or A-C 6702) is melted, and the other ingredients are blended with it. The mixture is then made into sheets by any suitable means such as extrusion or rolling into sheets of suitable thickness such as 0.25 mm, 0.45 mm, 0.50 mm, 0.67 mm, 0.73 mm, or 1.0 mm. The sheet is then cut into shapes suitable for application to dentures.

### Example II

| | A | B | C | D |
|---|---|---|---|---|
| | % | % | % | % |
| Ca/Zn AVE/MA Salt | 33.00 | 33.00 | | 20.00 |
| CMC | 20.00 | 20.00 | 53.00 | 33.00 |
| Mineral Oil | 11.97 | 5.98 | 11.97 | 5.30 |
| Microcrystalline Wax² W-835 | 23.46 | 35.19 | 23.50 | 35.25 |
| Flavors | | | | 0.5 |
| Sacharrin | 0.04 | 0.06 | | 0.08 |
| Colorants | | | | 0.10 |
| Silica | 0.57 | 0.29 | 0.57 | 0.29 |
| Petrolatum | 10.96 | 5.48 | 10.96 | 5.48 |

²Multiwax W 835 manufactured by Witco (Crompton, Sonneborn). The Multiwax W 835 may also be substituted with either Polyethylene A-C 1702 or Polyethylene A-C 6702.

In Example II, the Microcrystalline Wax W-835 (or Polyethylene AC 1702 or A-C 6702) and Petrolatum are melted, and the other ingredients are blended with it. The mixture is then made into sheets by any suitable means such as extrusion or rolling into sheets of suitable thickness such as 0.25 mm, 0.45 mm, 0.50 mm, 0.67 mm, 0.73 mm, or 1.0 mm. The sheet is then cut into shapes suitable for application to dentures.

### Example III

| | A | B | C | D | E | F | G |
|---|---|---|---|---|---|---|---|
| | % | % | % | % | % | % | % |
| Ca/Zn AVE/MA Salt | | | | 33.00 | 33.00 | 30.00 | 10.00 |
| CMC | 10.00 | 20.00 | 30.00 | 20.00 | 20.00 | | |
| AVE/MA Acid S-97 | | | | 1.00 | | | |
| Microcrystalline | 90.00 | 80.00 | 70.00 | 30.00 | 23.50 | 70.00 | 90.00 |
| Wax³ W-180 | | | | | | | |
| Flavors | | | | 0.50 | | | |
| Saccharin | | | | 0.16 | | | |
| Colorants | | | | 0.10 | | | |
| Petrolatum | | | | 15.24 | 23.50 | | |

³Multiwax W 180 manufactured by Witco (Crompton, Sonneborn).

The Microcrystalline Wax W-180 is melted, and the other ingredients are blended with it. The mixture is then made into sheets by any suitable means such as extrusion or rolling into sheets of suitable thickness such as 0.50 mm, 0.67 mm, 0.73 mm, or 1.0 mm. The sheet is then cut into shapes suitable for application to dentures.

In the above examples, all or part of the Ca/Zn AVE/MA salt may be substituted with Mg/Zn/Na AVE/MA salts and/or Ca/Na AVE/MA salts; all or part of the CMC may be substituted with HEC, Caraggeenan, and/or Karaya Gum; all or part of the Microcystalline Wax W-180 may be substituted with Microcrystalline Wax W-445 (also available from Witco); all or part of the Petrolatum may be substituted with Mineral Oil, and/or Polybutene; and/or the amount of each ingredient may also be increased or decreased by up to about 50%. Each of the above examples may be blended with each other prior to making into sheets; and/or used in conjunction with each other to form multilayer articles.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A denture adhesive article, comprising:
(a) a denture adhesive component, and
(b) a viscosity index improver,
wherein the denture adhesive article is preformed.

2. The denture adhesive article of claim 1, wherein said viscosity index improver is selected from the group consisting of polymethacrylates, olefin copolymers, hydrogenated styrene-diene copolymers, styrene polyesters, rubber, polyvinylchloride, nylon, fluorocarbon, polyurethane prepolymer, polyethylene, polystyrene, polypropylene, cellulosic resins, acrylic resins, microcrystalline wax, elastomers, poly(n-butyl vinyl ether), poly(styrene-co-maleic anhydride), poly(alkyl fumarate co-vinyl acetate), alkylated polystyrene, poly(t-butyl styrene), and combinations thereof.

3. The denture adhesive article of claim 2, wherein the viscosity index improver is in an amount from about 10.0% to about 90.0% by weight of the denture adhesive article.

4. The denture adhesive article of claim 1, wherein the viscosity index improver is selected from the group consisting of microcrystalline wax, polyethylene, rubber, elastomers, and mixtures thereof.

5. The denture adhesive article of claim 1, wherein the viscosity index improver is selected from the group consisting of microcrystalline wax, polyethylene, and mixtures thereof.

6. The denture adhesive article of claim 3, wherein the viscosity index improver is in an amount from about 40.0% to about 60.0% by weight of the denture adhesive article.

7. The denture adhesive article of claim 1, wherein the denture adhesive component is selected from the group consisting of cellulose, cellulose derivatives, starch, starch derivatives, saccharide, saccharide derivatives, polyethylene oxides, polyethylene glycols, polyvinyl alcohols, carrageenan, alginates, karaya gum, xanthan gum, guar gum, gelatin, algin, tragacanth, chitosan, acrylamide polymers, carboxypolymethylene, polyamines, polyquaternary compounds, polyvinylpyrrolidone, AVE/MA, salts of AVE/MA, mixed salts of AVE/MA, polymeric acids, polymeric salts, polyhydroxy compounds, and combinations thereof.

8. The denture adhesive article of claim 7, wherein the denture adhesive component is in an amount from about 10.0% to about 60.0% by weight of the denture adhesive article.

9. The denture adhesive article of claim 8, wherein the denture adhesive component is selected from the group consisting of AVE/MA, salts of AVE/MA, mixed salts of AVE/MA, sodium carboxymethylcellulose, and combinations thereof.

10. The denture adhesive article of claim 8, wherein the denture adhesive component is in an amount from about 20.0% to about 55.0% by weight of the denture adhesive article.

11. The denture adhesive article of claim 1, further comprising a water insoluble component, wherein the water insoluble component is selected from the group consisting of petrolatum, polyvinyl acetate, natural oils, synthetic oils, fats, silicone, silicone derivatives, dimethicone, silicone resins, hydrocarbons, hydrocarbon derivatives, polybutene, oleic acid, stearic acid, essential oils, caprilic/capric triglycerides, and combinations thereof.

12. The denture adhesive article of claim 1, wherein the denture adhesive article is flexible, nonaqueous, and/or bioerodible.

13. The denture adhesive article of claim 1, wherein the denture adhesive article comprises a homogeneous mixture of (a) and (b).

14. The denture adhesive article of claim 1, wherein the denture adhesive article has a single layer.

15. The denture adhesive article of claim 1, wherein the denture adhesive article has dry tack properties.

16. The denture adhesive article of claim 15, wherein the shear storage modulus G′ measured in dynes/cm² at a frequency of about 1 Hz at about 25° C. is from about 3×10⁶ to about 1×10⁹.

17. The denture adhesive article of claim 1, wherein said denture adhesive article further comprises at least one non-adhesive self-supporting substrate.

18. The denture adhesive article of claim 1, wherein said denture adhesive article has a normalized ooze amount of from about 0% to about 20%, or an ooze ratio of from about 0 to about 0.7.

19. The denture adhesive composition of claim 1, wherein the viscosity index improver has an average molecular weight of about 450 to about 800.

20. The denture adhesive composition of claim 1, wherein the viscosity index improver has about 30 to about 75 carbon atoms per molecule.

21. The denture adhesive composition of claim 1, wherein the viscosity index improver has a refractive index of about 1.435 to about 1.445.

22. The denture adhesive composition of claim 1, wherein the viscosity index improver has a melting point from about 60° C. to about 93° C.

23. The denture adhesive composition of claim 1, wherein the viscosity index improver has a closed cup flash point of about 260° C.

24. The denture adhesive composition of claim 1, wherein the viscosity index improver has a viscosity at 98.9° C. from about 10.2 mm²/s to about 25 mm²/s.

25. The denture adhesive composition of claim 1, wherein the composition has an ooze ratio from about 0.4 to about 0.8.

26. The denture adhesive composition of claim 1, wherein the viscosity index improver has a penetration value less than about 175 when measured using ASTM D-937.

27. The denture adhesive composition of claim 1, wherein the viscosity index improver has a penetration value less than about 100 when measured using ASTM D-1321.

28. The denture adhesive composition of claim 1, wherein the viscosity index improver has:
a. an average molecular weight of about 450 to about 800;
b. about 30 to about 75 carbon atoms per molecule;
c. a refractive index of about 1.435 to about 1.445;
d. a melting point from about 60° C. to about 93° C.;
e. a closed cup flash point of about 260° C.;
f. a viscosity at 98.9° C. from about 10.2 mm²/s to abut 25 mm²/s;
g. a penetration value less than about 175 when measured using ASTM D-937; and
h. a penetration value less than about 100 when measured using ASTM D-1321.

29. The denture adhesive composition of claim 1, wherein the viscosity index improver has:
a. an average molecular weight of about 450 to about 800;
b. about 30 to about 75 carbon atoms per molecule;
c. a refractive index of about 1.435 to about 1.445;
d. a melting point from about 60° C. to about 93° C.;
e. a closed cup flash point of about 260° C.;
f. a viscosity at 98.9° C. from about 10.2 mm²/s to about 25 mm²/s;
g. a penetration value less than about 175 when measured using ASTM D-937; or
h. a penetration value less than about 100 when measured using ASTM D-1321.

30. The denture adhesive composition of claim 29, wherein the viscosity index improver comprises a wax.

31. The denture adhesive composition of claim 30, wherein the viscosity index improver comprises microcrystalline wax.

32. The denture adhesive composition of claim 30, wherein the viscosity index improver comprises paraffin wax.

33. The denture adhesive composition of claim 30, wherein the viscosity index improver comprises a petroleum wax.

34. The denture adhesive composition of claim 29, wherein the viscosity index improver comprises n-alkanes.

35. The denture adhesive composition of claim 34, wherein the viscosity index improver comprises n-alkanes with greater than about 30 carbon atoms.

36. The denture adhesive composition of claim 1, wherein the composition is an article that can be dispensed from a tube.

37. The denture adhesive composition of claim 1, wherein the viscosity index improver is crystalline.

38. The denture adhesive composition of claim 37, wherein at least some of the denture adhesive component is at least partially coated by crystals of the viscosity index improver.

39. The denture adhesive composition of claim 1, wherein the composition has an instant viscosity ratio of at least about 0.25.

40. The denture adhesive composition of claim 1, wherein the composition has an instant viscosity ratio of at least about 0.30.

41. The denture adhesive composition of claim 1, wherein the composition has an instant viscosity ratio of at least about 0.35.

42. The denture adhesive composition of claim 1, wherein the composition is substantially free of petrolatum, polybutene, silicones, and/or polyvinylacetate.

43. The denture adhesive composition of claim 1, wherein the composition is bioerodible, non-aqueous, and/or a uniform mixture of a component dispersed within the viscosity index improver.

44. A denture adhesive article, comprising:
(a) from about 10.0% to about 60.0% of a denture adhesive component comprising AVE/MA, salts of AVE/MA, mixed salts of AVE/MA, or a combination thereof; and
(b) from about 10% to about 90% of a viscosity index improver comprising microcrystalline wax, polyethylene, rubber, elastomers, or a combination thereof;
wherein the denture adhesive article is preformed.

45. The denture adhesive composition of claim 44, wherein the denture adhesive component comprises a combination of a mixed salt of AVE/MA and carboxymethylcellulose.

46. The denture adhesive composition of claim 44, wherein the viscosity index improver comprises microcrystalline wax.

47. The denture adhesive composition of claim 44, wherein the composition has an ooze ratio from about 0 to about 0.4.

48. The denture adhesive composition of claim 44, wherein the viscosity index improver has:
a. an average molecular weight of about 450 to about 800;
b. about 30 to about 75 carbon atoms per molecule;
c. a refractive index of about 1.435 to about 1.445;
d. a melting point from about 60° C. to about 93° C.;
e. a closed cup flash point of about 260° C.;
f. a viscosity at 98.9° C. from about 10.2 mm²/s to abut 25 mm²/s;
g. a penetration value less than about 175 when measured using ASTM D-937; and/or
h. a penetration value less than about 100 when measured using ASTM D-1321.

49. The denture adhesive composition of claim 48, wherein the viscosity index improver comprises a wax.

50. The denture adhesive composition of claim 49, wherein the viscosity index improver comprises microcrystalline wax.

51. The denture adhesive composition of claim 49, wherein the viscosity index improver comprises paraffin wax.

52. The denture adhesive composition of claim 49, wherein the viscosity index improver comprises a petroleum wax.

53. The denture adhesive composition of claim 48, wherein the viscosity index improver comprises n-alkanes.

54. The denture adhesive composition of claim 53, wherein the viscosity index improver comprises n-alkanes with greater than about 30 carbon atoms.

55. The denture adhesive composition of claim 44, wherein the composition is substantially free of petrolatum, polybutene, silicones, and/or polyvinylacetate.

56. The denture adhesive composition of claim 44, wherein the composition is bioerodible, non-aqueous, and/or a uniform mixture of a component dispersed within the viscosity index improver.

57. A method of manufacturing a denture adhesive composition, the composition comprising:
a) from about 10.0% to about 60.0% of a denture adhesive component comprising AVE/MA, salts of AVE/MA, mixed salts of AVE/MA, carboxymethylcellulose, or a combination thereof; and
b) from about 10.0% to about 90.0% of a viscosity index comprising microcrystalline wax, polyethylene, rubber, elastomers, and combinations thereof; the method steps comprising:
i. mixing at least one of the denture adhesive components with the viscosity index improver at a temperature above about 10° C. below the melting point of the viscosity index improver; and
ii. subsequently cooling the composition to about 10° C. below the melting point of the viscosity index improver.

58. The method of claim 57, wherein the denture adhesive component comprises a mixed salt of AVE/MA and carboxymethylcellulose and the viscosity index improver comprises microcrystalline wax.

59. The method of claim 58, wherein only the carboxymethylcellulose is mixed with the viscosity index improver at a temperature above about 10° C. below the melting point of the viscosity index improver, and wherein the mixed salt of AVE/MA is added and mixed after cooling the composition to about 10° C. below the melting point of the viscosity index improver.

60. The method of claim 58, wherein the carboxymethylcellulose is mixed with the viscosity index improver at a temperature above about 10° C. below the melting point of the viscosity index improver, and the mixed salt of AVE/MA is then mixed with the carboxymethylcellulose and viscosity index improver before the cooling step.

61. The method of claim 58, wherein the mixed salt of AVE/MA is mixed with the viscosity index improver at a temperature above about 10° C. below the melting point of the viscosity index improver, cooling the mixed salt of AVE/MA and viscosity index improver to about 10° C. below the melting point of the viscosity index improver, and then adding the carboxymethylcellulose.

62. A denture adhesive kit comprising:
(a) a plurality of preformed denture adhesive articles comprising a safe and effective amount of a denture adhesive component; and
(b) an outer package or inner dispensing package having an upper denture indicia representing the placement of one or more articles only in the grooves of the denture.
